(19) United States
(12) Patent Application Publication
(10) Pub. No.: US 2002/0123456 A1
(43) Pub. Date: Sep. 5, 2002

(54) Methods of identifying agents affecting atrophy and hypertrophy

(76) Inventor: David J. Glass, Cortlandt Manor, NY (US)

Correspondence Address: Laura J. Fischer, Regeneron Pharmaceuticals, Inc., 777 Old Saw Mill River Road, Tarrytown, NY, 10591, US

(21) Appl. No.: 10/086,201

(22) Filed: Feb. 28, 2002

Related U.S. Application Data
(60) Provisional application No. 60/273,174, filed on Mar. 2, 2001.

(51) Int. Cl. A61K 31/00; G01N 33/53; G01N 33/567

(52) U.S. Cl. 514/1; 435/7.2

(57) ABSTRACT

The present invention provides a method for inhibiting atrophy or causing hypertrophy in muscle cells, by inhibiting the action of the phosphatase SHIP2. It further provides a method for inhibiting skeletal muscle atrophy or causing skeletal muscle hypertrophy in vertebrate animals, by inhibiting the Akt pathway through the inhibition of SHIP2. It also provides a method of identifying agents that may be used for inhibiting atrophy or causing hypertrophy in muscle cells, by screening for inhibitors of SHIP2 or inhibitors of the SHIP2 signaling pathway.

Throughout this application various publications are referenced. The disclosures of these publications in their entireties are hereby incorporated by reference into this application. This application claims priority to provisional application U.S. Application No. 60/273,174 filed Mar. 2, 2001.

### BACKGROUND OF THE INVENTION

A decrease in muscle mass, or atrophy, is associated with various physiological and pathological states. For example, muscle atrophy can result from denervation due to nerve trauma; degenerative, metabolic or inflammatory neuropathy, e.g. Guillian-Barré syndrome; peripheral neuropathy; or nerve damage caused by environmental toxins or drugs. Muscle atrophy may also result from denervation due to a motor neuropathy including, for example, adult motor neuron disease, such as Amyotrophic Lateral Sclerosis (ALS or Lou Gehrig's disease); infantile and juvenile spinal muscular atrophies; and autoimmune motor neuropathy with multifocal conductor block. Muscle atrophy may also result from chronic disease resulting from, for example, paralysis due to stroke or spinal cord injury; skeletal immobilization due to trauma, such as, for example, fracture, ligament or tendon injury, sprain or dislocation; or prolonged bed rest. Metabolic stress or nutritional insufficiency, which may also result in muscle atrophy, include inter alia the cachexia of cancer and other chronic illnesses including AIDS, fasting or rhabdomyolysis, and endocrine disorders such as disorders of the thyroid gland and diabetes. Muscle atrophy may also be due to a muscular dystrophy syndromes such as Duchenne, Becker, myotonic, fascioscapulohumeral, Emery-Dreifuss, oculopharyngeal, scapulohumeral, limb girdle, and congenital types, as well as the dystrophy known as Hereditary Distal Myopathy. Muscle atrophy may also be due to a congenital myopathy, such as benign congenital hypotonia, central core disease, nemalene myopathy, and myotubular (centronuclear) myopathy. Muscle atrophy also occurs during the aging process.

Muscle atrophy in various pathological states is associated with enhanced proteolysis and decreased production of muscle proteins. Muscle cells contain lysosomal proteases and cytosolic proteases. The cytosolic proteases include Ca²⁺-activated neutral proteases (calpains) and an ATP-dependent ubiquitin-proteasome proteolytic system. The lysosomal and cytosolic systems are capable of degrading muscle proteins in vitro, but less is known about their roles in proteolysis of muscle proteins in vivo. Some studies have reported that proteasome inhibitors reduce proteolysis in atrophying rat skeletal muscle (e.g. Tawa et al. (1997) J. Clin. Invest. 100:197), leading to suggestions that the ubiquitin-proteasome pathway has a role in the enhanced proteolysis. However, the precise mechanisms of proteolysis in atrophying muscle remain poorly characterized.

Insulin-like growth factor 1 (IGF-1), is a small protein growth factor that has been shown to cause hypertrophy when expressed in skeletal muscle (Coleman ME, DeMayo F, Yin K C, Lee H M, Geske R, Montgomery C, Schwartz R J Biol Chem 1995 May 19;270(20):12109-16). A signaling pathway that is activated in response to IGF-1 is the phosphatidylinositol 3-kinase (PI3K)/Akt pathway (PI3K/Akt). (Vanhaesebroeck, et al. (1997) TIBS 22:267; Toker and Cantley (1997) Nature 387:673; Rameh and Cantley (1999) J. Biol. Chem. 274:8347). PI3K causes phosphorylation of the cell membrane-bound molecule phosphatidylinositol 4,5-bisphosphate at the 3 position, resulting in phosphatidylinositol 3,4,5-trisphosphate. Akt then translocates to the cell membrane and binds to phosphatidylinositol 3,4,5-trisphosphate, where the Akt is activated.

Since the presence of phosphotidylinositol-3,4,5-trisphosphate [PtdIns(3,4,5)P3 or PI(3,4,5)P3] is necessary for activation of Akt, phosphatases which dephosphorylate phosphotidylinositol-3,4,5-trisphosphate may inactivate Akt. One such phosphatase is called SHIP2. This phosphatase removes the phosphate at the 5 position resulting in4,5 biphosphatidylinositol,phosphotidylinositol-3,4-bisphosphate, which does not bind Akt. Thus, activation of the SHIP2 phosphatase results in inactivation of Akt activity (Taylor V, Wong M, Brandts C, Reilly L, Dean N M, Cowsert L M, Moodie S, Stokoe D Mol Cell Biol 2000 Sep;20(18):6860-71).

Studies have shown that in response to increases in external load, skeletal muscle adapts by increasing muscle mass and protein content primarily through an increase in muscle fiber size which translates into greater tension output (Carson, J. A. Exercise Sport Science Rev. 25: 301-320 (1997)). However, the signaling pathways responsible for the regulation of muscle fiber size and muscle mass are unknown. Initial studies in cardiac hypertrophy (Molkentin, J. D. et al. Cell 93:215-228 (1998)) as well as early studies with skeletal muscle cells in vitro (Molkentin, J. D. et al. Cell 93:215-228 (1998); (Semarian, C. et al. Nature 400: 576-581 (1999) pointed toward a key role for the cyclosporin-inhibitable phosphatase known as calcineurin. However, recent attempts to determine the effects of calcineurin manipulation on skeletal muscle hypertrophy in vivo have been inconclusive (Semarian, C. et al. Nature 400: 576-581 (1999); Musaro, A. et al Nature 400: 581-585 (1999)). Recently, Baar and Esser (Baar, K. & Esser, K. Am. J. Physiol. Cell 45: C120-C127 (1999)) showed that phosphorylation of the 70-kD S6 protein kinase (p₇₀^{S6K}) increases in muscles with hypertrophy following a high-resistant exercise training protocol in rats. Additionally, IGF-1, which activates the PI3K/Akt pathway, is upregulated during load-induced hypertrophy (Carson, J. A. Exercise Sport Science Rev. 25: 301-320 (1997); Adams, G. R. & Haddad, G. R. J. Appl. Physiol. 81: 2509-2516 (1996)) and leads to muscle hypertrophy in transgenic mice overexpressing IGF-1 under the control of a skeletal-actin promoter (Coleman, M. E. et al J. Biol. Chem. 270: 12109-12116 (1995)). These observations are intriguing because p70^{S6K} and the 4E binding protein PHAS-I are key phosphoproteins involved in the regulation of protein translation initiation (Brunn, G. J. et al. Science 277: 99-101 (1997); Rhoads, R. E. J. Biol. Chem. 274: 30337-30340 (1999)). Moreover, activation of p₇₀^{S6K} correlates to increases in the translation of mRNA containing a 5′-TOP tract, including ribosomal proteins and the elongation factors (Vary, T. C. et al. Am. J. Physiol. Endocrinol. Metab. 278: E58-E64 (2000)). Phosphorylation of PHAS-I leads to its dissociation from eIF4E allowing eIF4E to bind to the 5′-cap structure of an mRNA and initiate the formation of the eIF4F complex. The phosphorylation/activation of p₇₀^{S6K} and PHAS-I is regulated, in part, by a protein kinase mTOR that is activated by the PI3K/Akt pathway and inhibited by rapamycin.

In accordance with the present invention, it has been discovered that activation of the PI3K/Akt pathway by inhibition of the phosphatase SHIP2 (SHIP2) results in increased hypertrophy and blocking of atrophy of skeletal muscle cells.

### SUMMARY OF THE INVENTION

The present invention provides a method of inhibiting skeletal muscle atrophy or increasing skeletal muscle hypertrophy, by inhibiting signaling pathways that lead to skeletal muscle atrophy and activating pathways that lead to skeletal muscle hypertrophy. Agents that activate the PI3K/Akt pathway are useful for preventing or reducing atrophy and/or causing hypertrophy in skeletal muscle cells. An inhibitor of the SHIP2 phosphatase is one such agent.

The preferred embodiment of the invention is a method of inhibiting atrophy or inducing hypertrophy in skeletal muscle cells comprising treating the cells with a specific inhibitor of SHIP2 phosphatase.

atrophyAnother embodiment of the invention is a method of inhibiting atrophy or inducing hypertrophy in skeletal muscle cells comprising treating the cells with a specific inhibitor of SHIP2 phosphatase which results in the activation of Akt.

Another embodiment of the invention is a method of inhibiting atrophy or inducing hypertrophy in skeletal muscle cells comprising treating the cells with a specific inhibitor of SHIP2 phosphatase which results in the activation of mTOR.

Another embodiment of the invention is a method of inhibiting atrophy or inducing hypertrophy in skeletal muscle cells comprising treating the cells with a specific inhibitor of SHIP2 phosphatase which results in the inhibition or antagaonism of the calcineurin pathway.

A preferred embodiment of the invention is a method of inhibiting atrophy or inducing hypertrophy in skeletal muscle cells comprising treating the cells with an inhibitor of the SHIP2 pathway.

Another preferred embodiment of the invention is a method of inhibiting atrophy or inducing hypertrophy wherein inhibitor of the SHIP2 pathway is muscle tissue specific.

Another preferred embodiment of the invention is a method wherein the inhibitor of the SHIP2 pathway causes phosphorylation of SHIP2.

In another embodiment of the invention, SHIP2 may be inhibited in non-muscle tissue.

In preferred embodiments of the invention the muscle cells are in a vertebrate animal having an atrophy-inducing condition, wherein the vertebrate animal is a chicken, rodent, rabbit, dog, cat, cow, horse, pig, sheep, primate, or human.

A further embodiment is one in which the vertebrate animal is treated prior to exposure to or onset of the atrophy-inducing condition.

Also preferred is an embodiment wherein the atrophy-inducing condition is immobilization, denervation, starvation, nutritional deficiency, metabolic stress, diabetes, aging, muscular dystrophy, bed rest, AIDS/HIV infection, cancer-associated cachexia or myopathy.

One embodiment is a method of identifying a test agent that inhibits muscle atrophy comprising:

(a) obtaining cells that express the following: 1) SHIP2; 2) an Akt substrate/reporter construct capable of measuring Akt substrate activation;

(b) subjecting the cells to a test agent;

(c) measuring the amount of Akt substrate activation in (a), wherein activation of the Akt substrate, or determination of Akt phosphorylation is used to identify a test agent that inhibits atrophy in muscle cells.

In addition to measuring the amount of AKT susbtrate activation, the invention also embodies the direct measurement of Akt avtivity and Akt association with phosphotidylinositol-3,4,5-trisphosphate.

In preferred embodiments of the method the cells are fibroblasts, muscle cells, myoblasts, or C2C12 cells.

Another embodiment of the invention is a method of inhibiting atrophy or inducingmuscle hypertrophy in skeletal muscle cells comprising treating the cells with an inhibitor of the SHIP2 pathway. The inhibitor may be a specific inhibitor of SHIP2, and/or may cause phosphorylation of Akt or be an activator of Akt.

An additional embodiment of the invention is a method of inhibiting atrophy in skeletal muscle cells comprising treating the cells with a muscle tissue-specific activator of the PI3K/Akt pathway. In all of the methods herein described the skeletal muscle cells may be in a vertebrate animal having an atrophy-inducing condition. Such vertebrate animal may be a chicken, rodent, rabbit, dog, cat, cow, horse, pig, sheep, primate, or human and may be treated prior to exposure to or onset of the atrophy-inducing condition. Such atrophy-inducing condition may be immobilization, denervation, starvation, nutritional deficiency, metabolic stress, diabetes, aging, muscular dystrophy, AIDS/HIV infection, cancer, bed rest or myopathy.

Another embodiment of the invention is a method of identifying a test agent that inhibits muscle atrophy or induces hypertrophy comprising obtaining cells that express SHIP2 and an Akt substrate/reporter construct capable of measuring Akt pathway activation; subjecting the cells to a test agent; measuring the amount of Akt pathway activation, wherein activation of the Akt pathway is used to identify a test agent that inhibits atrophy. In any of the methods of this invention, such cells may be fibroblasts, muscle cells, myoblasts, C2C12 cells, or any other cells capable of undergoing the specified method.

In another embodiment, the invention is a method of testing for phosphotidylinositol-3,4,5-trisphosphate hydrolysis or Akt binding to phosphotidylinositol-3,4,5-trisphosphate in a cell free assay utilizing standard techniques known in the art, including but not limited to ELISA or FRET.

Yet another embodiment of this invention is a method of reducing muscle atrophy or inducing muscle hypertrophy in skeletal muscle cells comprising treating the cells with an activator of the PI3K/Akt pathway. Such an activator may be a specific activator of the PI3K/Akt pathway, a specific activator of Akt, and may cause phosphorylation of Akt substrates, such as mTOR, forkhead, or GSK3.

The invention also embodies a method of reducing muscle atrophy or inducing muscle hypertrophy in skeletal muscle cells comprising treating the cells with a muscle tissue-specific activator of the PI3K/Akt pathway or inhibitor of the SHIP2 pathway or an inhibitor of SHIP2. Such skeletal muscle cells may be within a vertebrate animal, and such vertebrate animal may be a chicken, rodent, rabbit, dog, cat, cow, horse, pig, sheep, primate, or human.

Another embodiment of this invention is a cell comprising SHIP2 and an Akt substrate/reporter construct capable of measuring Akt substrate activation; and a SHIP2 antagonist for use in a method of inhibiting atrophy, inducing hypertrophy, activating the Akt pathway, interfering with the calcineurin pathway, or modulating Akt expression or activity.

An additional embodiment of this invention is a method of screening compounds useful for the treatment of muscle atrophy or detecting atrophy and related diseases and disorders comprising contacting a muscle cell expressing SHIP2 with a compound and detecting a change in the SHIP2 protein activity or the Akt pathway. This change may be measured by PCR, Taqman PCR, phage display systems, gel electrophoresis, yeast-two hybrid assay, Northern or Western analysis, immunohistochemistry, a conventional scintillation camera, a gamma camera, a rectilinear scanner, a PET scanner, a SPECT scanner, a MRI scanner, a NMR scanner, or an X-ray machine. The change in SHIP2 protein activity may be detected by detecting a change in the interaction of SHIP2 with one or more proteins, by detecting a change in the interaction of Akt with another protein, or by detecting a change in the level of one or more of the proteins in the Akt pathway. Such cells may be of skeletal muscle origin, may be cultured cells, or may be obtained from or may be within a transgenic organism. Such transgenic organisms include, but are not limited to a mouse, rat, rabbit, sheep, cow or primate.

Another embodiment of the invention is a method of detecting muscle atrophy in an animal comprising measuring SHIP2 in a patient sample.

Another embodiment of the invention is a method of modifying SHIP2 activity or activating Akt by gene therapy techniques. Such techniques are know in the field.

An additional embodiment of this invention is a method of treating illnesses, syndromes or disorders associated with muscle atrophy comprising administering to an animal a compound that modulates SHIP2 or the Akt pathway such that symptoms are alleviated. Such animal may be a mammal or a human.

Yet another embodiment of the invention is a method of identifying a test agent capable of inhibiting muscle atrophy or inducing muscle hypertrophy in vitro comprising contacting a mixture containing SHIP2 and phosphatidylinositol 3,4,5-trisphosphate with a test agent; and measuring the ability of SHIP2 to mediate the conversion of phosphatidylinositol 3,4,5-trisphosphate. Such measuring may be accomplished either by measuring the release of the phosphate at the “5” position, or by determining the amount of residual phosphatidylinositol 3,4,5-trisphosphate. Such measuring may also be accomplished by fluorescence, PCR, Taqman PCR, phage display systems, gel electrophoresis, yeast-two hybrid assay, Northern or Western analysis, immunohistochemistry, a conventional scintillation camera, a gamma camera, a rectilinear scanner, a PET scanner, a SPECT scanner, a MRI scanner, a NMR scanner, an X-ray machine, or any other means known in the art. Such test agent is a carbohydrate, a lipid, a protein, a salt, a nucleic acid, a small molecule, or any other organic or inorganic molecule.

### BRIEF DESCRIPTION OF THE FIGURES

FIGS. 1A-1C: Photograph of myotubes transfected with either a control vector, a vector which directs expression of SHIP2, or a vector which directs expression of a dominant negative mutant form of SHIP2. Myotube atrophy is caused by expression of SHIP2 while hypertrophy is induced by expression of a dominant negative mutant form of SHIP2. C2C12 myoblasts are transfected with either a control vector that directs expression of Green Fluorescent Protein (EGFP) (FIG. 1A), with a vector that directs the expression of both the SHIP2 phosphatase and Green Fluorescent Protein (SHIP2 IRES EGFP) (FIG. 1B), or with a vector that directs the expression of a dominant negative mutant form of the SHIP2 phosphatase, and Green Fluorescent Protein (FIG. 1C). Over-expression of SHIP2 results in thinner myotubes as compared to control (FIG. 1B). Over-expression of the dominant negative mutant form of SHIP2 results in hypertrophic myotubes (FIG. 1C).

FIG. 2: Expression of SHIP2 blocks Akt activity while expression of a dominant negative mutant form of SHIP2 increases Akt activity. Protein lysates were prepared from the cells pictured in FIGS. 1A-1C. Akt was immuno-precipitated from the lysates, and a standard Akt activity assay was performed in vitro, using a kit obtained from Cell Signaling(™). Briefly, immuno-precipitated Akt is incubated with a substrate (GSK3) and the substrate is then analyzed by an immuno-blot using an antibody specific to the phosphorylated form of GSK3. As shown in FIG. 2, expression of SHIP2 blocks Akt activity, as there is an almost complete inhibition of GSK3 phosphorylation. In contrast, expression of the dominant negative mutant form of SHIP2 potentiates Akt activity.

FIG. 3: Muscle hypertrophy is not blocked by CsA. a, Weight of the rat heart, expressed as percentage change from control, after daily treatment with the β2-adrenergic agonist clenbuterol (3 mg kg−1, subcutaneously) for 14 d (filled columns) or daily treatment with clenbuterol and cyclosporin for 14 d (open columns) (10 rats per group). b, Calcineurin phosphatase activity measured in plantaris muscle lysates from control rats (Con.), control rats treated with cyclosporin for 4 d (Con+CsA), 4-d compensatory hypertrophy rats (CH), and 4-d CH rats treated with cyclosporin (CH+CsA). The total amount of calcineurin was similar between groups as measured by western blotting. c, Cross-sections of the rat plantaris muscle stained with an anti-MyHC slow antibody. Groups were control (Con.), 14 d compensatory hypertrophy (CH) and 14 d CH treated daily with cyclosporin (CH+CsA). d, Weight of the rat plantaris muscle, expressed as percentage change from control, after 14 or 30 d of compensatory hypertrophy with vehicle (CH) or cyclosporin treatment (CH+CsA) (10 rats per group). e, Cross-sectional area of muscle fibers in the rat plantaris muscle of control (Con.), 14 d compensatory hypertrophy (CH) or 14 d compensatory hypertrophy plus cyclosporin treatment (CH+CsA) (five rats per group). Muscle fibers were classified as slow or fast on the basis of immunohistochemical staining with anti-MyHC slow and fast antibodies. f, Percentage of muscle fibers expressing slow MyHC in the plantaris muscle of control (Con.), 14 d compensatory hypertrophy (CH) or 14 d compensatory hypertrophy plus cyclosporin treatment (CH+CsA) (five rats per group). g, Weight of mouse plantaris muscle, expressed as percentage change from control, after 7 d of compensatory hypertrophy with vehicle (CH) or FK506 treatment (CH+FK506) (10 mice per group).

FIG. 4: Muscle hypertrophy is associated with activation of the Akt/mTOR pathway and is blocked by rapamycin. a, Western blots of native and phosphorylated Akt in the plantaris during compensatory hypertrophy (CH). Each lane represents 200 μg of total protein extracted from a pool of three plantaris muscles after control (Con., lanes 1), 3 d CH (lanes 2), 7 d CH (lanes 3) or 14d CH (lanes 4). For each group, duplicate lanes represent different pools of plantaris muscles. b, Western blots of native and phosphorylated Akt, phosphorylated GSK-3β and p70s6k in the rat plantaris. The increase in Akt and GSK-3β phosphorylation measured after 14 d (lane 2) of compensatory hypertrophy (CH/−) was not inhibited by daily injections of rapamycin (CH/Rap; lane 3). The p70s6k gel shift observed after 14 d of compensatory hypertrophy (lane 2) was inhibited by daily injections of rapamycin (CH/Rap; lane 3). Each lane represents 200 μg (Akt and GSK-3β) or 25 μg (p70s6k) of total protein extracted from a pool of three plantaris muscles. c, The specific activity of GSK-3β was determined by 32P incorporation into phospho-glycogen synthase peptide 2 in the immune complex. The GSK-3β activity was measured in the plantaris of control (Con.); 14 d compensatory hypertrophy (CH) and 14 d CH treated daily with rapamycin (CH+RAP) are shown. d, The specific activity of p70s6k was determined by 32P incorporation into 40S ribosomes in the immune complex. The p70s6k activity was measured in the plantaris of control (Con.); 14 d compensatory hypertrophy (CH) and 14 d CH treated daily with rapamycin (CH+Rap) are shown. e, PHAS-I/4E-BP1 bound to eIF4E after 14 d compensatory hypertrophy (CH) or 14 d CH treated daily with rapamycin (CH+Rap). After correction for the amounts of eIF4E recovery, the results were expressed as a percentage of the respective controls and are means ±range for two experiments. f, eIF4G bound to eIF4E after 14 d compensatory hypertrophy (CH) or 14 d CH plus rapamycin (CH+Rap). After correction for the amounts of eIF4E recovery, the results were expressed as a percentage of the respective controls and are means ±range for two experiments. g, Weight of the rat plantaris muscle, expressed as percentage change from control, after 7 or 14 d of compensatory hypertrophy with vehicle (CH) or rapamycin treatment (CH+Rap) (10 rats per group). Asterisk indicates significant difference from CH group (P<0.05). h, Cross-sectional area of muscle fibers in the rat plantaris muscle of control (Con.), 14 d compensatory hypertrophy (CH) or 14 d compensatory hypertrophy plus rapamycin treatment (CH+Rap) (five rats per group). Muscle fibers were classified as slow or fast on the basis of immunohistochemical staining with anti-MyHC slow and fast antibodies. Asterisk indicates significant difference from CH group (P<0.05).

FIG. 5: Recovery of muscle weight after HLS is blocked by rapamycin, but not cyclosporin. a, Western blots of native and phosphorylated Akt and p70s6k in the medial gastrocnemius (MG) after HLS and recovery (Rec.). Each lane represents 200 μg (Akt) or 25 μg (p70s6k) of total protein extracted from a pool of three MG muscles after control (Con., lanes 1), 14 d HLS (lanes 2) or 14 d HLS followed by 7 d of recovery (lanes 3). For each group, duplicate lanes represent different pools of MG muscles. b, PHAS-1/4E-BP1 bound to eIF4E in MG muscle after 14 d of HLS or 14 d of HLS followed by 7 d of recovery (Rec.). After correction for eIF4E recovery, the results were expressed as percentages of the control. Means ±range for two experiments are presented. c, Hindlimb muscle weights, expressed as percentage changes from HLS, for the medial gastrocnemius (MG), plantaris (Pl.) and soleus (Sol.) muscles. Muscles were taken after control (con), 14 d of HLS, 14 d of HLS followed by 7 d recovery (Rec.), and 14 d of HLS followed by 7 d of recovery plus treatment with rapamycin (Rec.+RAP) (10 rats per group). Asterisk indicates significant difference between recovery and Rec.+RAP groups (P<0.05). d, Hindlimb muscle weights, expressed as percentage changes from 14-d HLS values, for the medial gastrocnemius (MG), plantaris (Pl.) and soleus (Sol.) muscles. Muscles were taken after control (Con.), 14 d of HLS, 14 d of HLS followed by 7 d of recovery (Rec.), and 14 d of HLS followed by 7 d of recovery plus treatment with cyclosporin (Rec.+CsA) (10 rats per group).

FIG. 6: Expression of activated Akt in normal and denervated muscle fibres induces hypertrophy. a, b, Representative cross-sections of the TA muscle from control and denervated mice. Normal and denervated TA muscles were injected with either a control (EGFP) or an activated Akt (c.a. Akt-EGFP) plasmid. Transfected fibres were identified 7 d after injection and electroporation on the basis of their expression for EGFP. The distribution of cross-sectional areas of EGFP-expressing fibres taken from three muscles under each condition (a, control/EGFP, control/c.a. Akt-EGFP; b, denervation/EGFP, denervation/c.a. Akt-EGFP) were plotted as frequency histograms. The mean ±s.e.m. is given for each group. c, Mean cross-sectional area of muscle fibres in normal mouse TA 7 d after transfection with HSA/EGFP (Con.) or HSA/SHIP2-HA (SHIP2). d, Mean cross-sectional area of transfected muscle fibres in control and compensatory hypertrophied rat plantaris muscle. The plantaris was surgically overloaded and injected with either HSA/EGFP (CH) or HSA/SHIP2-HA (CH/SHIP2) followed by electroporation. Muscle fibre size was determined 7 d after surgery and plasmid injection. Control muscles were analyzed 7 d after injection with HSA/EGFP (Con.).

FIG. 7 Schematic overview of a signaling network downstream of the growth factor IGF-1, emphasizing the primary role of the PI(3)K/Akt/mTOR cascade, linking receptor tyrosine kinase derived signals to growth regulatory mechanisms. The data also suggest possible cross-talk between the interacting kinases Akt and mTOR, and the NFAT-C1 transcription factor

FIG. 8: Signaling pathways activated by IGF-1 or the calcium ionophore A23187 (Ca-I) in C2C12 differentiated myotubes. a, Opposing effects of Ca-I and IGF-1 on the activation of the transcription factor NFAT-C1 in differentiated myotubes; immunoblot analysis. Ca-I (1.0 μM, 15 min) induces a dephosphorylation (compare lanes 1 and 3, top panel) and translocation (lane 3, bottom panel) of NFAT-C1 from the cytoplasm into the nuclear fraction. Treatment with CsA (5 μM) before stimulation with Ca-I shows that dephosphorylation and translocation requires calcineurin (lane 4). IGF-1 stimulation (10 ng ml-1) leads to a hyperphosphorylation of NFAT-C1 (lane 5). In an independent experiment, treatment of the myotubes simultaneously with IGF-1 and Ca-I inhibits the dephosphorylation and translocation of NFAT-C1 induced by Ca-I (compare lanes 8 and 10). b, Stimulation with IGF-1 activates the PI(3)K/Akt/mTOR and PI(3)K/Akt/GSK3 pathways independently of calcineurin blockade; immunoblot analysis. Stimulation of myotubes with IGF-1 (10 ng ml-1, 15 min) increased the phosphorylation of Akt, p70S6K, GSK3, PHAS-1/4E-BP1 and Erk1/2 (lane 3) compared with control (lane 1); treatment with Ca-I (1.0 μM, 15 min), an activator of calcineurin, had no effect on these kinases (lane 2). CsA (5 μM, 15 min), a calcineurin inhibitor, did not inhibit IGF-1 (lane 4). c, Treatment of myotubes with kinase inhibitors for PI(3)K (10 μM LY294002 (LY)) and mTOR (2 ng ml-1 rapamycin (RAP)) before stimulation with IGF-1 (10 ng ml-1, 15 min); immunoblot analysis. Akt and GSK3 phosphorylation depend on PI(3)K activity and are independent of mTOR. p70S6K phosphorylation and kinase activity, assayed on the 40S ribosomal subunit (40rsu-p), is blocked by the inhibitors LY294002 and rapamycin. Rapamycin did not block GSK3 phosphorylation. An inhibitor of Mek1/2, PD98059 (PD), showed that the IGF-1-induced phosphorylation of Akt, GSK3 and p70S6K does not require MAP-kinase kinase/Erk kinase activity.

FIG. 9. IGF-1 induces skeletal myotube hypertrophy via the PI(3)K/Akt/mTOR kinase cascade, independently of calcineurin activity. a-d, Calcineurin inhibition blocks myotube differentiation but is not required for hypertrophy. a, C2C12 myoblasts, at confluence on day 0, then differentiated in 5 μM CsA, analyzed for morphological effects at day 4 after fusion. Untreated myotubes are shown (Con.). b, Myotubes treated at day 2 after fusion with either or both of CsA (5 μM) or IGF-1 (10 ng ml-1), analyzed at day 4 of differentiation. IGF-1 caused hypertrophy; CsA did not block hypertrophy. c, Myotubes treated with the calcium ionophore A23187 (Ca-I) at 1 μM. The ionophore did not stimulate myotube hypertrophy. d, C2C12 cells genetically-engineered for inducible expression of constitutively active calcineurin (c.a. calcin), induced with 2 μg ml-1 doxycycline (DOX) at day 2 after differentiation. Calcineurin did not induce hypertrophy. e, Pharmacological inhibitors of PI(3)K and mTOR kinase on IGF-1-induced skeletal myotube hypertrophy. Myotubes were treated at day 2 after fusion with the PI(3)K inhibitor LY294002 (LY; 10 μM), the mTOR inhibitor rapamycin (RAP, 2 ng ml-1), IGF-1 (10 ng ml-1) and with a combination of IGF-1 and each inhibitor, and analyzed for morphological effects at day 4. Blockade of the PI(3)K/Akt/mTOR pathway inhibited hypertrophy. f-I, Comparison of the phenotypes of C2C12 myotubes expressing Akt mutants, SHIP2 mutants, constitutively active p70S6K (c.a. p70S6K) and dominant- negative GSK3β (d.n. GSK3β). f, Expression of wild-type Akt (w.t. Akt), c.a. Akt and kinase-inactive Akt (k.i. Akt) shows that c.a. Akt increased myotube size, whereas k.i. Akt diminished it. g, Comparison of EGFP (control), wild-type SHIP2 (w.t. SHIP2) and dominant-negative SHIP2 (d.n. SHIP2): SHIP2 decreased myotube size; d.n. SHIP2 increased myotube size. h, Constitutively active p70S6K (c.a. p70S6K) increased myotube size. i, Expression of dominant-negative GSK3β (d.n. GSK3β) resulted in a markedly increased myotube size. EGFP is shown as control. j, Effect of Ara-C, an inhibitor of DNA replication, on IGF-1-mediated hypertrophy. C2C12 myotubes were treated at day 2 after induction of differentiation with IGF-1 (10 ng ml-1) or a combination of Ara-C (10 μM) and IGF-1 (10 ng ml-1), and analysed the next day. Untreated myotubes are shown (CON). Ara-C did not block IGF-1-mediated hypertrophy. Quantification of myotube diameters is shown. The average number of nuclei per myotube is presented for each condition in the bottom row.

FIG. 10: Direct or indirect Akt activation results in p70S6 kinase activation. a, Phosphorylation of p70S6K is induced by constitutively active Akt (c.a. Akt), shown as a control for Akt activity in the cells transfected in FIG. 3f. Immunoblot analysis demonstrates the expression of the haemagglutinin-tagged c.a. Akt (HA c.a. Akt), compared with the control cell line. b, Expression of wild-type SHIP2 decreases IGF-induced Akt activity, as measured by immunoblotting with a phosphospecific antibody for Akt, which detects activated Akt. c, Expression of dominant negative SHIP2 (d.n. SHIP2) increases p70S6 activity, as determined by a shift in the ratio of p70S6 kinase phosphorylated bands, demonstrating a shift to the higher-molecular-mass, active form of p70S6 kinase in the presence of d.n. SHIP2. d, Expression of c.a. Akt in serum-starved differentiated myotubes increases the phosphorylation of NFAT-C1 and diminishes the ability of calcineurin to dephosphorylate NFAT-C1. Immunoblot analysis shows that stimulation of C2C12 with the calcium ionophore A23187 (Ca-I, 1.0 μM, 15 min) causes dephosphorylation and nuclear translocation of NFAT-C1 (control, lanes 1 and 2). Expression of c.a. Akt (lanes 3 and 4) leads to a hyperphosphorylation of NFAT-C1 (lane 3) and diminishes the Ca-Iinduced dephosphorylation and translocation of NFAT-C1 (compare lanes 2 and 4).

### DETAILED DESCRIPTION OF THE INVENTION

Muscle atrophy can be caused by the enhanced proteolysis of muscle proteins, resulting in a decrease in muscle mass. In addition, muscle atrophy can be caused by a diminished synthesis of proteins, resulting in a decrease in muscle mass.

It has been discovered in accordance with the present invention that when skeletal muscle cells contain and express the active SHIP2 phosphatase, the resultant cells appear atrophic in comparison to control skeletal muscle cells.

It has also been discovered in accordance with the present invention that when skeletal muscle cells contain and express a dominant negative form of the SHIP2 phosphatase, which inhibits SHIP2 phosphatase activity or competes for substrate binding, the resultant cells appear hypertrophic in comparison to control skeletal muscle cells. (A “dominant negative” mutant form of a signaling molecule is a mutant that is capable of inhibiting normal signaling of that molecule.)

Accordingly, Applicants have discovered that specific activators of the PI3K/Akt pathway are useful for reducing or preventing atrophy or causing hypertrophy in skeletal muscle cells. As used here, “specific activators” of the PI3K/Akt pathway are those that result in activation of a substrate within the PI3K/Akt pathway; specifically, in accordance with the present invention, inhibition of SHIP2 or the SHIP2 pathway results in activation of the Akt pathway. In addition applicants have discovered that when SHIP2 is electroporated in muscles, an increased load, which in other muscles would cause an activation of Akt and result in hypertrophy, does not result in hypertrophy as it is blocked by the overexpression of SHIP2.

According to the invention, specific inhibitors of SHIP2 or the SHIP2 pathway are agents that may be used to decrease and/or prevent atrophy in mammals having a condition, such as those described herein, in which skeletal muscle atrophy is occurring. According to this embodiment, atrophying skeletal muscle cells, or vertebrate animals having a condition as described above in which muscle cells are atrophying, are treated with a specific inhibitor of SHIP2 so as to prevent or decrease muscle cell atrophy. Such treatment may be utilized prophylactically prior to the onset of muscle atrophy or after such condition has manifested itself. Vertebrate animals include any species containing skeletal muscle and a backbone, and includes chickens, rodents, rabbits, dogs, cats, cows, horses, pigs, sheep, primates, and humans, preferably humans.

In another embodiment, specific inhibitors of SHIP2 or the SHIP2 pathway are agents that may be used to cause hypertrophy in skeletal muscle cells. Further, such specific inhibitors of SHIP2 or the SHIP2 pathway may be used to cause muscle hypertrophy in vertebrate animals having conditions, such as those described herein, in which skeletal muscle atrophy is anticipated. In some settings, such as in animals farmed for meat production, such agents might be used to increase meat production, and other agricultural uses wherein increased hyertophy or decreased atrophy would be beneficial(e.g. stronger, larger or faster farm animals or recreation animals).

The invention further provides for compositions, including therapeutic compositions, comprising a specific inhibitor of the SHIP2 pathway in a carrier that may include excipients, diluents or other compounds. Such compositions may be administered systemically or locally. Any appropriate mode of administration known in the art may be used including, but not limited to, intravenous, intrathecal, intraarterial, intranasal, oral, subcutaneous, intraperitoneal, or by local injection or surgical implantation. Sustained release formulations are also provided for.

The activity of the compositions of the invention in vertebrate animals may be assessed using experimental animal models of disorders in which muscle atrophy is present. For example, the activity of the compositions may be tested for their effect in the hindlimb immobilization model described herein in Example 2 infra. Alternatively, the activity of the compositions may be assessed using experimental animals in which hypertrophy can be measured. For example, the activity of the compositions may be tested for their effect on muscles undergoing exercise-induced hypertrophy, or compensation-induced hypertrophy. Alternatively, the muscle may be assessed in control animals as compared to animals treated with the experimental compositions, to determine if the treated animals exhibit skeletal muscle hypertrophy as a result of their treatment. Data obtained from cell culture assays and animal studies can be used in formulating a range of dosages for use in vertebrate animals, including humans. The dosage of the compositions of the invention should lie within a range of serum circulating concentrations with little or no toxicity. The dosage may vary within this range depending on the dosage form employed and the route of administration.

To screen for specific inhibitors of SHIP2 or the SHIP2 pathway in a cell-based assay, a cell which expresses SHIP2, such as a myoblast or myotube, but also any other cell which expresses SHIP2, including a fibroblast, may be used to determine if a reporter construct containing an Akt phosphorylation site or downstream component (“Akt substrate/reporter construct”) becomes phosphorylated when contacted with a test agent (Akt would be used as a readout for SHIP2 inhibition, because Akt is activated when SHIP2 is inhibited). One skilled in the art will recognize that other components in the Akt pathway may also be used in place of Akt, provided the appropriate substrate/reporter construct is present. For example, the assay may comprise a more downstream member of the Akt pathway.

Examples of substrates that are phosphorylated by Akt include GSK3 (glycogen synthase kinase 3) (Cross, et al. (1995) Nature 378: 785, BAD; Datta, et al. (1997) Cell 91, 231-41; del Peso, et al. (1997) Science 278: 687), the transcription factor “forkhead” (Brunet, (1999) Cell 96: 857; Kops, et al. (1999) Nature 398: 630); and mTOR.

To assess the specificity of the inhibition of SHIP2-activity, other phosphatases can be assessed, including the related phosphatase SHIP1. In some cases, it may be acceptable to have agents that cross-react between SHIP1 and SHIP2. In these cases, more distantly related phosphatases, such as SKIP or PTEN could be used to determine specificity.

Examples of reporter constructs that may be used to assess contact or enzymatic activity between SHIP2 and a substrate include those used in FRET-based assays. Inhibition, as used herein, would be defined as an amount below the level normally expressed by the cell in the absence of the agent.

In a preferred embodiment of the invention, cells are created that comprise SHIP2, and an Akt substrate/reporter construct capable of measuring Akt pathway activation. Test agents that may be utilized to inhibit atrophy or cause hypertrophy are identified as those that cause SHIP2 or SHIP2 pathway inhibition, or subsequent Akt pathway activation.

Cells useful for expressing Akt and SHIP2 and their associated substrate/reporter constructs include non muscle cells, as well as any and all muscle cells that can be maintained in culture and that can be engineered to express a heterologous nucleic acid. The cells may be primary cultures or established cell lines. Suitable muscle cells include myoblasts, for example the C2C12 cell line as described in Bains, et al. (1984) Mol. Cell. Biol. 4:1449, the disclosure of which is incorporated herein by reference. Other suitable muscle cells include Sol8 cells, described by Glass et al. (1997) Proc. Natl. Acad. Sci. USA 16:8848, and L6 cells, described by Ringentz et al. (1978) Exp. Cell Res. 113:233, the disclosures of which are incorporated herein by reference.

The SHIP2, Akt and reporter construct nucleic acids under the control of suitable transcriptional and translational regulatory sequences can be introduced into the cell by methods known in the art including, for example, transformation, transfection, infection, transduction and injection. The expression vector containing SHIP2 and Akt substrate/reporter construct nucleic acids under the control of suitable promoters is introduced into cells by known methods, for example liposome-mediated transfection, calcium phosphate-mediated transfection, DEAE-dextran transfection, naked DNA transfection, microinjection, electroporation, retroviral-mediated infection, adenoviral-mediated infection, or adeno-associated viral-mediated infection. The reporter construct nucleic acids can be introduced into the cell stably or transiently. Methods for introducing heterologous nucleic acids into eukaryotic cells are described in numerous laboratory manuals including, for example, DNA Cloning: A Practical Approach, vols. I-III (1985) Glover, ed., IRL Press Limited, Oxford, and Sambrook et al. (1989) Molecular Cloning: A Laboratory. Manual, 2^{nd} edition, Cold Spring Harbor, N.Y.

In a preferred embodiment, the nucleic acid is inserted into a retroviral vector, for example, as described by Pear et al. (1993) Proc. Natl. Acad. Sci. USA 90:8392, incorporated herein by reference. In this embodiment, the viral LTR promoter controls the transcription of the nucleic acid. The vector is transiently transfected into a retroviral packaging line, and the resulting recombinant virus which contains the nucleic acid is harvested, as described by Pear et al., id. The recombinant virus is then used to infect myoblasts as described by Hoffman et al. (1996) Proc. Natl. Acad. Sci. USA 93:5185, incorporated herein by reference. C2C12 cells, as an example of skeletal muscle cells expressing SHIP2 and Akt, can be maintained in the undifferentiated state by growing them in tissue culture media containing at least 10% fetal calf serum, or they can be differentiated into skeletal muscle myotubes by growing them in media containing 2% horse serum. The necessary tissue culture methods are known to those of ordinary skill in the art. C2C12 cells are described in Bains et al. (1984) Mol. Cell Biol. 4:1449, incorporated herein by reference.

In methods for the identification of an agent that inhibits muscle cell atrophy, or causes hypertrophy, the agent may be contacted with the cell comprising SHIP2, Akt and the substrate/reporter constructs by methods known in the art. For cells in culture or cells obtained from transgenic organisms, the cell may be contacted with the agent by, for example, direct application. The agent may be modified or contained in a delivery vehicle to facilitate entry into the cell. The agent may be isolated and purified, or it may be present in a sample or composition to be subjected to further isolation and purification subsequent to a positive result in the present method. For example, the agent may be contained in a cell lysate, conditioned cell culture media, or a library of synthetic or naturally occurring compounds. For muscle cells present in a transgenic organism, the cells may be contacted with the agent by delivering the agent by methods known in the art, for example by ingestion, parenteral administration, or direct application to tissue surfaces, and may be present in a composition comprising a carrier or diluent. Agents that may be tested in the method of the present invention include, for example, organic and inorganic molecules such as proteins, peptides, lipids, carbohydrates, nucleic acids, including antisense, metals, salts, and so on.

Test agents identified above may be assessed for their ability to cause hypertrophy or reduce atrophy in cultured muscle cells. The amount of atrophy in cells may be measured by quantitation of cell diameter, protein amount, or by activation of the Akt pathway member p70^{S6K} which stimulates protein synthesis. In vertebrate animals, muscle atrophy may be measured as described in Example 2 herein.

In yet another preferred embodiment, components of the SHIP2 pathway, and the Akt pathway are expressed in an in vitro assay, such that agents can be screened to determine their capability of inhibiting SHIP2 in vitro. Inhibition of SHIP2 activity in vitro can also be assessed by binding of tagged or purified Akt to phosphotidylinositol-3,4,5-trisphosphate in vitro in the presence of a SHIP 2+/−test agent or by binding of another phosphotidylinositol-3,4,5-trisphosphate-specific reporter molecule.

The presence of activated PI3K/Akt substrates can also be assessed using antibodies specific for these proteins.

In another embodiment, the present invention provides a method of identifying an agent that inhibits atrophy or causes hypertrophy in muscle cells, comprising preparing an in vitro assay for SHIP2, contacting the SHIP2 protein with an agent to be tested; and screening for agents which inhibit the SHIP2 protein. According to this embodiment, an assay could be developed to take advantage of the fact that SHIP2 is a phosphatase, and when activated it dephosphorylates the phosphatidylinositol 3,4,5-trisphosphate. Thus, SHIP2 protein could be contacted with an appropriate substrate and a test agent, in an in vitro phosphatase assay, and the substrate could then be analyzed for whether it had become dephosphorylated by SHIP2. To assure specificity, other phosphatidylinositiol phosphatases, such as SKIP, would also be assayed with the same test agent, to assure that the agent causes inactivation of SHIP2 specifically.

The methods of the present invention are useful for the identification of agents that prevent atrophy or cause hypertrophy in muscle cells by specifically inhibiting SHIP2 or the SHIP2 pathway. The agents identified by the present methods are useful for the treatment and prevention of muscle atrophy and for causing muscle hypertrophy.

In addition to the embodiments described supra, a tissue-specific mechanism of inhibiting SHIP2 or the SHIP2 pathway would also be very valuable. Applicants have demonstrated that activation of Akt can lead to an increase in protein synthesis, and can block muscle atrophy. Current agents such as IGF-1 activate the PI3K/Akt pathway in skeletal muscle, but also in many other tissues, since the receptor for IGF-1 is ubiquitous. Therefore, an agent whose receptor is tissue-specific, or which can be engineered to act in a tissue-specific fashion, such as by specific introduction of the agent into muscle, or by joining the agent to a second moiety which confers tissue specificity, would be useful in blocking muscle atrophy, or in inducing muscle hypertrophy. A tissue-specific inhibitor of SHIP2 or the SHIP2 pathway would have the additional benefit of avoiding side effects caused by lack of specificity, such as cardiac muscle hypertrophy, for example. The following non-limiting examples serve to further illustrate the present invention.

### EXAMPLES

### Example 1

### The Effect of Inhibition of SHIP2 in Muscle Cells

The role of the SHIP2 pathway in C2C12 myoblasts and differentiated myotubes was examined by genetic manipulation. Genetic manipulation of C2C12 cells was accomplished by the transfection of vectors capable of expressing a gene of interest as well as the green fluorescent protein (GFP) reporter gene. This enabled isolation of rare clones of transfected cells expressing desired levels of the gene of interest, using standard fluorescence activated cell sorter (FACS) technology. Expression of the transgenes was confirmed by standard immuno-blotting. HA-epitope tagged, constitutively active SHIP2 (Pesesse X, Deleu S, De Smedt F, Drayer L, Erneux C. (1997) Biochem Biophys Res Commun. Oct 29;239(3):697-700.) was subcloned into a bi-cistronic vector consisting of the MCK promoter (Jaynes, et al. (1988) Mol. Cell. Biol. 8:62) and an IRES-EGFP cassette (Clontech). Subconfluent C2C12 myoblasts were transfected by calcium phosphate-mediated co-transfection (Specialty Media, Inc.). Transfection was performed as described previously (Glass, et al. (1996) Cell 85:513). Flow cytometry and cell sorting were carried out on a Cytomation MoFlo (Fort Collins, Colo.) high-speed cell sorter. Laser excitation was 130 mw at 488 n. Fluorescence emission was collected through a 530/540 nm band pass filter for GFP. For sorting, cells were collected at a sort rate of 25,000 cells/second. The collected myoblasts were then grown to confluence, and differentiated by standard techniques into “myotubes”, which are multi-nucleated muscle cells, similar to actual muscle fibers.

Expression of GFP alone did not alter the C2C12 differentiation process. Vector-driven expression of a dominant negative mutant form of SHIP2 caused a hypertrophic phenotype, resulting in thickened multinucleated myotubes. The effect was even more pronounced than that observed previously by the Applicants, in which a dominant negative form of Raf is expressed by C2C12 cells (See PCT International Application No. PCT/US00/17173, filed Jun. 22, 2000, in the name of Regeneron Pharmaceuticals, Inc., which is incorporated in reference in its entirety herein). Thus, genetic manipulation of the PI3K/Akt pathways reveals that stimulating the PI3K/Akt pathway by inhibition of the SHIP2 pathway promotes the hypertrophic phenotype.

Biochemical evaluation of the cells expressing SHIP2 or dominant negative mutant form of SHIP2 demonstrated regulation of the PI3K/Akt pathways. Protein lysates were prepared from myotubes expressing either a control vector, SHIP2, or a dominant negative mutant form of SHIP2. Akt was immuno-precipitated from these lysates and used in an in vitro kinase assay using GSK3 as a substrate. Expression of SHIP2 resulted in reduced Akt activity, as measured by an immunoblot detecting phosphorylation of GSK3, a substrate of Akt. Expression of the dominant negative mutant form of SHIP2 resulted in increased Akt activity, relative to control.

### Example 2

### Animal Model For Atrophy

To test for muscle atrophy, the ankle joint of rodents (mice or rats) are immobilized at 90 degrees of flexion. This procedure induces atrophy of the muscles with action at the ankle joint (e.g. soleus, medial and lateral gastrocnemius, tibilias anterior) to varying degrees. A reproducible amount of atrophy can be measured in hindlimb muscles over a 14-day period.

The immobilization procedure may involve either casting (mice) or pinning the ankle joint (rats). Rodents are anesthetized with ketamine/xylazine and the right ankle joint is immobilized. In rats, a 0.5 cm incision is made along the axis of the foot, over the heel region. A threaded screw (1.2×8 mm) is then inserted through the calcaneous and talis, into the shaft of the tibia. The wound is closed with skin glue. In mice, the ankle joint is fixed at 90 degrees with a light weight casting material (VET-LITE) around the joint. The material is soaked in water and then wrapped around the limb. When the material dries it is hard, but light in weight.

At seven and 14 days following the immobilization, animals are anesthetized and killed by cervical dislocation. The tibialis anterior (TA), medial gastrocnemius (MG), and soleus (Sol) muscles are removed from the right (immobilized) and left (intact) hindlimbs, weighed, and frozen at a fixed length in liquid nitrogen cooled isopentane. A cohort of control animals which are the same weight and age as the experimental animals are also killed and the muscles removed, weighed and frozen. The amount of atrophy is assessed by comparing the weight of the muscles from the immobilized limb with the weight of the muscles from the control animals. Further assessment of atrophy will be done by measuring muscle fiber size and muscle tension output.

### Example 3

### The Calcineurin Pathway was not Activated During Hypertrophy in vivo, and Inhibitors of Calceneurin, Cyclosporin A and FK506 did not Blunt Hypertrophy

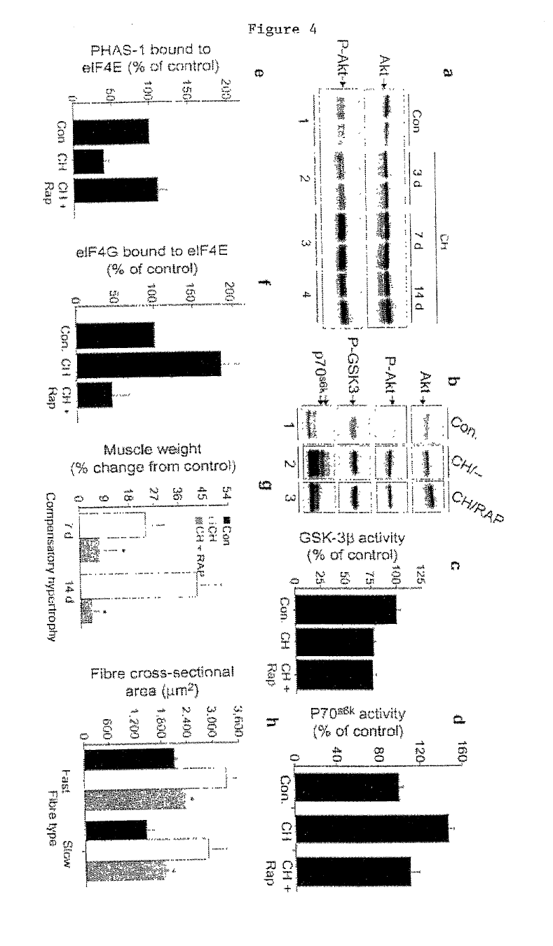

Initial studies in cardiac hypertrophy, as well as early studies with skeletal muscle cells in vitro pointed towards a key role for the cyclosporin-inhibitable phosphatase known as calcineurin (Semarian, C. et al. Nature 400, 576-581 (1999); Musaro, A. et al. Nature 400, 581-585 (1999); Molkentin, J. D. et al. Cell 93, 215-228 (1998).) We first examined the role of the calcineurin pathway in a model of compensatory muscle hypertrophy. When a fast-twitch skeletal muscle is subjected to a chronic workload increase by removing functionally synergistic muscles, the muscle compensates by increasing fibre size and muscle weight, as well as by switching fibres to a slowtwitch phenotype (Adams, G. R. & Haddad, G. R. J. Appl. Physiol. 81, 2509-2516 (1996); Roy, R. R. et al. J. Appl. Physiol. 83, 280-290 (1997).) The switch to a slow fibre phenotype has recently been suggested to be under the control of a calcineurin-dependent pathway, although the Ras/MAPK (mitogen-activated protein kinase) pathway has also been implicated (Naya, F. J. et al. J. Biol. Chem. 275, 4545-4548 (2000); Murgia, M. et al. Nature Cell Biol. 2, 142-147 (2000)). Functional overload of the rat plantaris muscle was induced by surgically removing the soleus and gastrocnemius muscles. Cyclosporin A (CsA) was given at a dosage (15 mg kg-1, subcutaneously) sufficient to block completely the cardiac hypertrophy induced pharmacologically by the β2-adrenergic agonist clenbuterol (FIG. 3a) and which inhibited calcineurin activity in control skeletal muscle (FIG. 3b, first two columns). Treatment with CsA was unable to prevent compensatory hypertrophy of the plantaris at 7, 14 or 30 days after the surgical overload, as shown by the increases in muscle weight (FIG. 3d) and fibre size (FIGS. 3c, e). Furthermore, treatment with CsA had no effect on the percentage of fibres expressing slow myosin heavy chain (MyHC) after compensatory hypertrophy (FIGS. 3c, f) or the shifts in MyHC expression (data not shown). Consistent with the lack of a role for calcineurin induction during compensatory hypertrophy, calcineurin activity was not increased but rather decreased in hypertrophying muscle (FIG. 3b, third column). Finally, treatment with FK506, a calcineurin inhibitor that functions through the binding of FK506-binding protein 12 (FKBP12), was unable to prevent the increase in muscle weight associated with compensatory hypertrophy (FIG. 3g).

### Example 4

### Akt/mTOR Pathway was Upregulated During Hypertrophy and Downregulated During Nuscle Atrophy

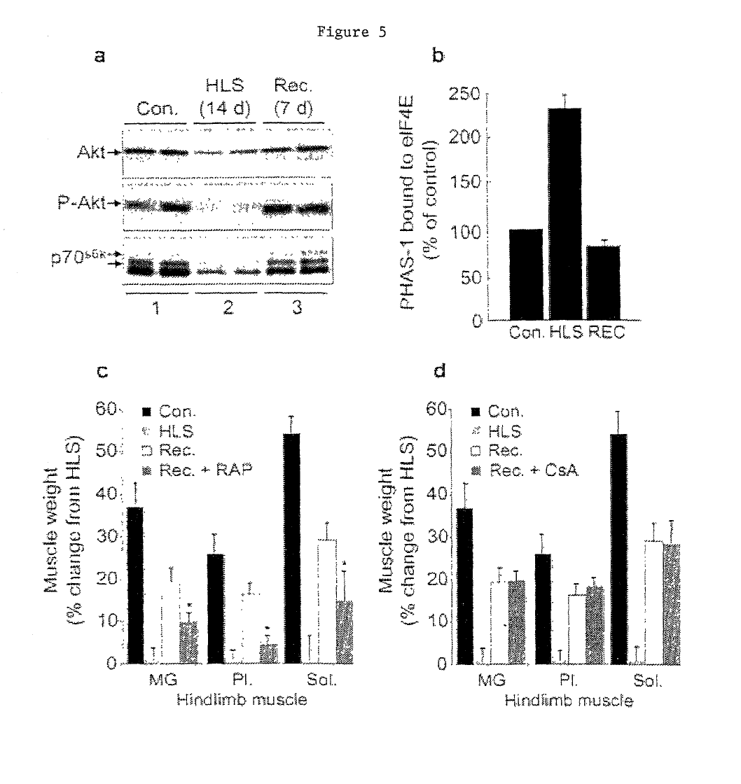

We have found that hypertrophy of myotubes induced in vitro by insulin-like growth factor 1 (IGF-1) depended on a pathway initiated by PtdIns-3-OH kinase (PI(3)K) and the PtdIns-regulated kinase Akt, which in turn led to activation of the rapamycin-sensitive kinase known as mTOR, whose downstream targets, p70S6K and PHAS-1/4E-BP1, have been shown to promote protein synthesis through increases in translation initiation and elongation. (Terada, N. et al. Proc. Natl Acad. Sci. USA 91, 11477-11481 (1994); Brunn, G. J. et al. Science 277, 99-101 (1997); Rhoads, R. E. J. Biol. Chem. 274, 30337-30340 (1999); Lin, T. -A. et al. Science 266, 653-656 (1994); Lin, T. -A. & Lawrence, J. C. Jr J. Biol. Chem. 271, 30199-30204 (1996)). To obtain initial evidence for a role of the Akt pathway during muscle hypertrophy in vivo, we examined Akt phosphorylation in the model of compensatory hypertrophy of the plantaris described above. The amount of Akt, and more importantly the phosphorylation state representing activated Akt, increased throughout the hypertrophy process. By 14 days, the total amount of Akt increased fourfold over control, whereas the level of phosphorylated/activated form increased ninefold in the hypertrophying plantaris (FIG. 4a) as determined by densitometry. Akt seems to promote protein synthesis in several ways. For example, Akt phosphorylates glycogen synthase kinase 3β (GSK-3β), leading to its inhibition and the upregulation of protein synthesis. More extensively characterized is the ability of Akt to activate p70S6K and PHAS-1/4E-BP1, presumably through mTOR, although this is controversial and has not been proved in vivo. Phosphorylation of p70S6K leads to its activation and to the promotion of protein synthetic pathways, whereas phosphorylation of PHAS-1/4E-BP1 releases it from within an inhibitory complex with the translation initiation factor eIF4E, thereby permitting the binding of eIF4E to eIF4G and promoting translation initiation. Consistent with a crucial role for the Akt pathway during muscle hypertrophy in vivo, the above Aktinducible events were noted in hypertrophying plantaris. That is, in addition to Akt phosphorylation, GSK-3β phosphorylation and inhibition were noted (FIGS. 4b, c). Furthermore, downstream targets of mTOR were clearly involved, as p70S6K was inducibly phosphorylated and activated (FIGS. 4b, d), whereas PHAS-1/4E-BP1 was released from eIF4E (FIG. 4e), allowing its binding to eIF4G (FIG. 4f).

The above findings indicate that the Akt pathway and its downstream targets are activated during muscle hypertrophy in vivo. To begin to determine the role of these activations, we used treatment in vivo with rapamycin, a quite specific inhibitor of one important Akt target, mTOR. Rapamycin, a selective blocker of mTOR, blocked hypertrophy in all models without causing atrophy in control muscles Rapamycin binds to its intracellular receptor, forming a complex that then binds to and inhibits mTOR activity (Schmeizie, T. & Hall, M. N. Cell 103, 253-262 (2000).). Consistent with the biochemical site of rapamycin action was our observation that treatment with rapamycin in vivo did not alter the phosphorylation or activity of Akt itself or of its mTORindependent target GSK-3β (FIGS. 4b, c), but instead specifically blocked targets known to be downstream of mTOR12-17, such as the phosphorylation and activation of p70S6K (FIGS. 4b, d) and the release of inhibition of eIF4E by PHAS1/4E-BP1 (FIGS. 4e, f). Most importantly, treatment with rapamycin in vivo almost completely prevented the hypertrophic increases in plantaris muscle weight (FIG. 4g) and fibre size (FIG. 4h) at 7 and 14 days. Treatment with rapamycin produced no non-specific effects; that is, there was no effect on body weight or the baseline weight of non-overloaded hindlimb muscles in these animals. Further, rapamycin given to control (surgically untreated) adult animals for 14 days had no effect on body weight or muscle weight (data not shown). In addition to the above results from rats, similar results were obtained when this hypertrophy model was performed in mice (data not shown).

These data demonstrate that during adaptive hypertrophy in adult animals, Akt and its downstream targets, GSK-3β p70S6K and PHAS-1/4E-BP1, are phosphorylated; moreover, the finding that specific inhibition of mTOR with rapamycin leads to a 95% blockage of hypertrophy indicates that the activation of mTOR and its targets, p70S6K and PHAS-1/4E-BP1, are necessary for adaptive hypertrophy.

### Example 5

### Recovery of Muscle Weight After Atrophy Through Disuse

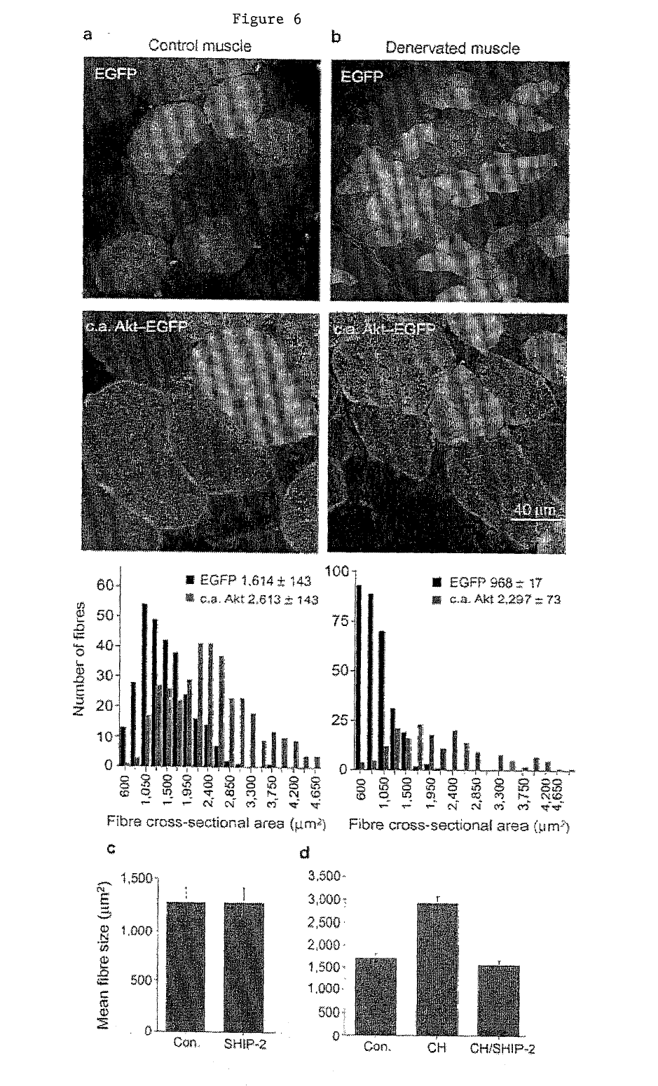

To extend the above findings to other models of skeletal muscle hypertrophy, we next focused on the recovery of muscle weight after atrophy through disuse. Although increases in the load on a muscle result in muscle hypertrophy, decreased use of a muscle results in atrophy, as occurs when the hindlimbs of rodents are suspended and no longer work against gravity (Thomason, D. B., Herrick, R. E., Surdyka, D. & Baldwin, K. M. J. Appl. Physiol. 63, 130-137 (1987)). Atrophied hindlimb muscles, when reloaded after a period of disuse, undergo a hypertrophic recovery of their original muscle weight over the course of weeks. Depending on the particular hindlimb muscle examined, muscles from rats undergoing hindlimb suspension (HLS) for 14 days showed a 25-55% loss of muscle weight (FIGS. 5c, d). After release from HLS, the atrophied muscles were reloaded and hypertrophied, achieving a 15-20% recovery in muscle weight after 7 days of reloading (FIGS. 5c, d). As seen in the previous hypertrophy model, the activation of Akt and its targets was correlated with the trophic state of the muscle during both atrophy through disuse and hypertrophy on recovery. That is, Akt protein and phosphorylation levels decreased markedly during the atrophy accompanying HLS (FIG. 5a, second set of lanes), as did the activation state of p70S6K (FIG. 5a, second set of lanes), whereas the PHAS-1/4E-BP1 inhibitory complex with eIF4E increased (FIG. 5b, second column). These changes reverted during recovery of the muscle when it was removed from HLS (FIG. 5a, third set of lanes, and FIG. 5b, third column).

### Example 6

### Genetic Activation of the AKT/mTOR Pathway was Sufficient to Cause Hypertrophy and Prevent Atrophy in vivo, Whereas Genetic Blockade of Their Pathway Blocked Hypertrophy in vivo

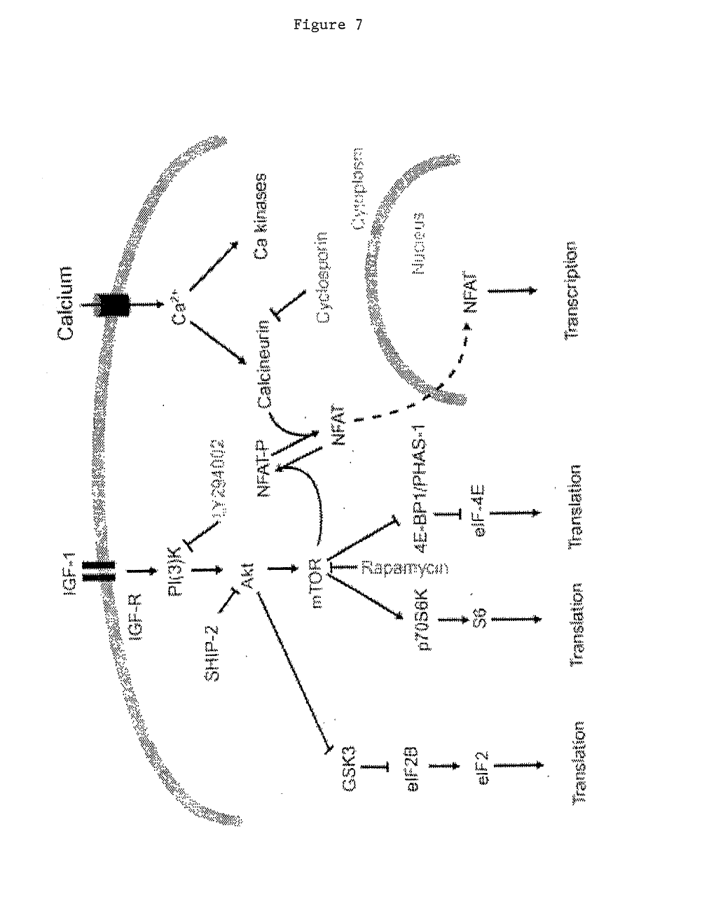

As with the compensatory hypertrophy model described above, not only was activation of the Akt/mTOR pathway correlated with muscle trophic changes in the HLS model, but, more importantly, treatment with the mTOR-inhibitor rapamycin during recovery from suspension markedly blocked the muscle growth observed during the 7-day reloading period (FIG. 6c). Also consistent with the findings in the compensatory hypertrophy model was our observation that CsA had no effect on the recovery of muscle weight during the 7-day reloading period (FIG. 6d).

Taken together, the above findings indicate that the Akt/mTOR pathway is activated in, and requisite for, muscle hypertrophy in vivo. To test the hypothesis that activation of mTOR and its downstream targets was not only required for muscle fibre hypertrophy but could actually trigger it, we injected a genetic construct designed to express a constitutively active form of Akt (c.a. Akt) into the tibialis anterior (TA) muscle of adult mice (Eves, E. M. et al. Mol. Cell. Biol. 18, 2143-2152 (1998)). We have demonstrated that the over-expression of c.a. Akt in vitro leads to phosphorylation of p70S6K and PHAS-1/4E-BP1 and to marked hypertrophy of myotubes. The c.a. Akt was fused to enhanced green fluorescent protein (EGFP) and over-expressed under the control of the human skeletal actin promoter (Brennan, K. J. & Hardeman, E. C. J. Biol. Chem. 268, 719-725 (1993)).

Muscle fibres that had taken up and expressed the activated Akt were identified on the basis of their expression for EGFP (FIG. 6). Those fibres expressing EGFP showed no signs of injury or regeneration. Muscle fibre size was determined in normal TA muscles 7 days after injection with either a plasmid expressing c.a. Akt-EGFP or EGFP alone. In three EGFP-treated and three c.a. Akt-treated normal TA muscles, cross-sectional area was determined in all fibres expressing EGFP (˜100 fibres per muscle). Mean fibre size was significantly larger in fibres overexpressing c.a. Akt (2,613±148 μm2) than in fibres expressing the control EGFP plasmid (1,615±143 μm2) (FIG. 6a). The distribution of fibre sizes shifted to the right, with the range in fibre sizes increasing from 634−3,873 μm2 in control muscles to 781−5,347 μm2 in muscles overexpressing c.a. Akt. Changes in fibre size were evident within 48 h of transfection with c.a. Akt (data not shown). Further, as an additional control, haemagglutinin (HA)-tagged c.a. Akt under the control of the cytomegalovirus (CMV) promoter was tested for its ability to promote fibre growth. Fibres expressing c.a. Akt were identified on the basis of immunohistochemical staining for the HA tag and were found to be larger than control at 2 and 14 days after transfection (data not shown).

Given that over-expression of c.a. Akt could induce hypertrophy of normal fibres, we tested whether c.a. Akt could prevent muscle fibre atrophy. The sciatic nerve was cut, resulting in denervation and atrophy of the TA muscle. Concurrent with the denervation, TA muscles were injected with either the c.a. Akt-EGFP or the EGFP plasmid. At 7 d after the denervation, denervated muscle fibres expressing c.a. Akt were significantly larger than denervated fibres expressing EGFP alone (2,297±73 μm2 versus 968±17 μm2) (FIG. 6b). Denervated fibres had a fibre size distribution that was shifted to the left relative to control (410−2,289 μm2), whereas denervated fibres expressing c.a. Akt had a fibre size distribution that was more similar to control (624−5,716 μm2). To confirm that c.a. Akt was acting via the mTOR pathway, we showed that the hypertrophy induced by c.a. Akt in normal and denervated fibres could be blocked by the concurrent administration of rapamycin (data not shown). Finally, genetic activation of Akt with a constitutively active PI(3)K construct could also prevent denervation-induced muscle atrophy (data not shown).

### Example 7

### Over Expression of SHIP2

Given that genetic activation of Akt could induce hypertrophy and prevent atrophy, we attempted to block endogenous Akt activation genetically by overexpressing the inositol 5-phosphatase SHIP2, which would independently confirm the above data from the use of rapamycin to block hypertrophy pharmacologically. We have found that the over-expression of SHIP2 in C2C12 myotubes inhibited IGF-1-induced Akt activation and blocked myotube hypertrophy. Over-expression of SHIP2 in normal TA muscles had no effect on fibre size (FIG. 6c), which was consistent with the response of rapamycin in normal muscles. In contrast, over-expression of SHIP2 in fibres of the rat plantaris during compensatory hypertrophy completely blocked the hypertrophy response (FIG. 6d), which was again consistent with the ability of rapamycin to block compensatory hypertrophy pharmacologically.

Skeletal muscles modify their size throughout life in response to changes in external loads and neural activity. The signaling pathways responsible for regulating cell size in adult skeletal muscle are poorly understood. Our work demonstrates that adaptive hypertrophy of adult skeletal muscle seems to be crucially regulated by the activation of the Akt/mTOR pathway and its downstream targets, p70S6K and PHAS-1/4E-BP1. Although our data reveal that maintenance of muscle weight in mature animals is not dependent on the Akt/mTOR pathway (because neither rapamycin nor SHIP2 produced a loss of muscle weight in adult rodents), this pathway seems to be crucial in load-induced hypertrophy of adult muscle and the recovery of weight after atrophy. Our data go further to show that genetic activation of Akt is sufficient to induce hypertrophy in vivo and can oppose the mechanisms responsible for producing muscle atrophy by preserving muscle fibre size.

### Discussion of Examples 1-7

Contrary to previous reports, our data suggest that activation of a calcineurin signaling pathway is not crucial for load-induced hypertrophy of skeletal muscle or the switch to expression of slow MyHC. (Dunn, S. E., Burns, J. L. & Michel, R. N. J. Biol. Chem. 274, 21908-21912 (1999). Dunn, S. E., Chin, E. R. & Michel, R. N. J. Cell Biol. 151, 663-672 (2000).) Our conclusions are based on the findings that cyclosporin was unable to block hypertrophy after 7-30 days of daily administration and that calcineurin activity decreased, as opposed to increased, during the hypertrophy process. These conclusions are in disagreement with those of Dunn et al., who reported that CsA blocked load-induced hypertrophy when delivered at a dose of 25 mg kg-1 twice daily. However, those authors observed a significant decrease in the amount of hypertrophy only at 30 days after the surgical overload, but not at 7 or 14 days while hypertrophy was ongoing and already prominent. Thus, the inability of Dunn et al to block hypertrophy with CsA while it was continuing seems consistent with our conclusion that the calcineurin pathway is not required for the hypertrophy process. Moreover, the late effects of CsA in their hands probably reflect a general toxic effect of long-term, high-dose CsA administration because overall body weight significantly decreased in their long-term-treated animals. The recent findings that tenfold over-expression of activated calcineurin in muscle does not lead to muscle hypertrophy or additional growth after surgical overload, and that treatment with cyclosporin does not prevent IGF-1-mediated hypertrophy further supports the conclusion that calcineurin is not involved in a crucial signaling pathway that is necessary for adaptive hypertrophy of muscle fibres in adult rodents.

Our findings are consistent with previous suggestions that overload-induced hypertrophy is due to increases in translational capacity and/or translational efficiency because these are processes that indeed seem to be regulated by p70S6K and PHAS-1/4E-BP1 (Carson, J. A. Exercise Sport Science Rev. 25, 301-320 (1997); Baar, K., Blough, E., Dineen, B. & Esser, K. Exercise Sport Science Rev. 27, 333-379 (1999)). It has been postulated that increases in protein synthesis during compensatory hypertrophy are initially derived through increases in translational efficiency followed later by changes in translational capacity through the addition of myonuclei. Indeed, increases have been found in the number of myonuclei per fibre after compensatory overload, and irradiation studies have suggested that satellite-cell incorporation is crucial for compensatory hypertrophy of the rat EDL muscle. However, muscle hypertrophy can occur in the absence of satellite cell proliferation/fusion into myofibres (Roy, R. R., Monke, S. R., Allen, D. L. & Edgerton, V. R. J. Appl. Physiol. 87, 634-642 (1999); Rosenblatt, J. D., Yong, D. & Parry, D. J. Muscle Nerve 17, 608-613 (1994); Wong, T. S. & Booth F. W. J. Appl. Physiol. 69, 1718-1724 (1990). Lowe, D. A. & Always, S. E. Cell Tiss. Res. 296, 531-539 (1999); Baar, K. & Esser, K. Am. J. Physiol. Cell 45, C120-C127 (1999)). The mechanisms regulating muscle fibre size are obviously complex, and the degree to which changes in translation efficiency and/or satellite cell proliferation contribute to hypertrophy require further study.

The findings that p70S6K and PHAS-1/4E-BP1 are activated during hypertrophy and that their activation is blocked by rapamycin suggest that mTOR is a crucial regulator of muscle fibre size in adult animals. This regulation of mTOR in vivo is likely to be mediated via Akt/protein kinase B, which we demonstrated was activated during skeletal muscle hypertrophy and was sufficient to induce the hypertrophy process when introduced genetically. Recent genetic manipulation in Drosophila of PI(3)K, Akt and p70S6K have indicated a role of the PI(3)K/Akt/mTOR pathway in the regulation of cell size, in that inactivation of this pathway leads to smaller cells but not fewer cells. The role of p70S6K during postnatal growth has also been investigated in mice. Mice with a homozygous disruption of the p70S6K/p85S6K gene have lower body weights and organ weights relative to wild-type littermates. Additional support for the role of PI(3)K in muscle growth is provided by a recent study by Shoi et al., who demonstrated that cardiac-specific overexpression of a constitutively active PI(3)K in mice led to hearts that were larger than normal, primarily because of an increase in the size of the cardiomyocytes. Interestingly, Murgia et al. found that expression of an activated Ras that stimulates PI(3)K led to enhanced regeneration of injured muscle fibres. Further, Baar and Esser demonstrated that phosphorylation of p70S6K increases during high resistance exercise training and muscle hypertrophy in rats. Lastly, our findings in vivo demonstrate that overexpression of activated Akt leads to an increase in muscle fibre size. Activated Akt was capable not only of increasing the size of fibres in normal muscles but also of preserving muscle fibre size in muscles undergoing atrophy. Taken together, these data suggest that activation of mTOR via PI(3)K/Akt might serve as a crucial regulator of muscle fibre growth in vivo.

### Materials and Methods For Examples 3-7

Procedures in vivo.

Female Sprague-Dawley rats (250-275 g) were anaesthetized with ketamine/xylazine (50/10 mg kg-1, intraperitoneally); with the use of aseptic surgical techniques the soleus, medial gastrocnemius and lateral gastrocnemius muscles were removed bilaterally, producing a functional overload on the plantaris muscles. In some instances, plasmid DNA (100 μl) was injected into the muscle immediately after the procedure. The hindlimbs of adult female SD rats (225-250 g) were unloaded by suspending their tails with a tail-traction bandage as described29. At the appropriate times, rats were killed and hindlimb muscles were removed, weighed and frozen in isopentane cooled with liquid nitrogen. Weight-matched surgically untreated rats served as controls.

The TA muscle was isolated in adult C57BL/6 mice under isoflurane (2-2.5%) anaesthesia. In some mice the right sciatic nerve was isolated in the mid-thigh region and cut, leading to denervation of the lower limb muscles. Plasmid DNA (100 μg) was injected (30 μl) into the muscle along the length of the fibres with the use of a Hamilton syringe. Immediately after the plasmid injection, electric pulses were applied by two stainless steel pin electrodes placed on each side of the isolated muscle belly. Square-wave electric pulses (16 pulses with a duration of 20 ms at a frequency of 1 Hz) were delivered by an ECM 830 electroporation unit (BTX, San Diego, Calif.) at a field strength of 125 V cm-1. All procedures were done in accordance with guidelines set by the Institutional Animal Care and Use Committee.

Drug Administration in vivo.

Animals were randomized to treatment or vehicle groups so that the mean starting body weights of each group were equal. Drug treatment began on the day of surgery or on the first day of reloading after the 14-day suspension. Rapamycin was delivered once daily by intraperitoneal injection at a dose of 1.5 mg kg-1, dissolved in 2% carboxymethylcellulose. CsA was delivered once daily by subcutaneous injection at a dose of 15 mg kg-1, dissolved in 10% methanol and olive oil. FK506 was delivered once daily via subcutaneous injection at a dose of 3 mg kg-1, dissolved in 10% ethanol, 10% cremophor and saline.

Transfection in vivo. Constructs used encoded the following: (1) myristoylated, c.a. Akt (refs 22, 36) fused in frame at the 3• end to the gene encoding EGFP (Clontech), and subcloned into an expression vector containing the human skeletal actin (HSA) promoter23; or (2) the gene encoding EGFP alone, subcloned into the same expression vector containing the HSA promoter. The c.a. Akt-EGFP fusion protein was tested in C2C12 myotubes, and mediated the activation of p70s6k and PHAS1/4E-BP1 (data not shown), as expected for c.a. Akt. As an additional control, the myristoylated, HA-tagged c.a. Akt was subcloned into a vector consisting of the CMV promoter. Human inositol 5-phosphatase SHIP2 was cloned from an Origene library. SHIP2 was HA-tagged and subcloned into an expression vector containing the human skeletal actin promoter.

Calceneurin-Phophatase Assay

Plantaris muscles were homogenized on ice for 15 s in 10 ml of buffer containing 20 mM Tris-HCl pH 7.6, 50 mM NaCl, 1 mM EDTA, 1 mM phenyl methylsulphonyl fluoride (PMSF), 10 μg ml-1 leupeptin, 0 μg ml-1 aprotinin and 0.1% Nonidet P40. Lysates were cleared by centrifugation at 11,000 g for 15 min at 4° C. The supernatant fractions were then desalted in G-25 Sephadex columns (Boehringer Mannheim) equilibrated in lysis buffer. The total phosphatase activity (serine/threonine phosphatase assay system; Promega) in the lysates was determined in the presence of calmodulin (250 μg ml-1) and calculated from the difference in absorbance read in the presence and absence of both phosphopeptide substrate and okadaic acid. Phosphate release was measured at 30° C. for 2 min

Western Blots.

Muscles were homogenized at 4° C. in RIPA lysis buffer (1% Nonidet P40, 0.5% sodium deoxycholate, 0.1% SDS in 50 mM NaCl, 20 mM Tris-HCl pH 7.6) containing 1 mM PMSF, 10 μg ml-1 aprotinin, 10 μg ml-1 leupeptin, 5 mM benzamidine, 1 mM EDTA, 5 mM N-ethylmaleimide, 50 mM NaF, 25 mM •-glycerophosphate, 1 mM sodium orthovanadate, 100 nM okadaic acid and 5 nM microcystin LR. Homogenates were clarified by centrifugation at 12,000 g for 20 min before determination of protein concentration by bicinchoninic acid assay (Pierce Chemical Co.). SDS-PAGE was performed on 7.5% gels prepared with an acrylamide:bisacrylamide ratio of 100:1. Western blots were revealed with enhanced chemiluminescence (Renaissance; NEN). Antibodies against anti-p70s6k (C-18; Santa Cruz) and Akt (NEB) were used to detect protein expression levels. Antibodies against Akt phosphorylated on Ser 473 (NEB) and GSK-3β/β phosphorylated on Ser 21/9 (NEB) were used to detect the catalytically activated form of the kinase.

Kinase Assay of p70s6k.

Protein A-agarose beads (Bio-Rad; 0.1 ml of serum per ml of packed beads) were incubated at 23° C. for 60 min with nonimmune serum or antisera against p70s6k. The beads were then washed five times with PBS (145 nM NaCl, 4 mM KCl, 10 mM sodium phosphate pH 7.4) and once with homogenization buffer. Samples of extract (100 μl) were incubated with beads (10 μl) for 60 min at 4° C. with constant mixing and then washed twice (0.5 ml of homogenization buffer per wash) and suspended in 100 μl of homogenization buffer. To measure p70s6k activity, immune complexes were incubated with 10 μl of solution containing 50 mM sodium β-glycerophosphate (pH 7.4), 14 mM NaF, 10 mM MgCl2, 1 mM dithiothreitol, 9 μM cAMP-dependent protein kinase inhibitory peptide, 20 μM calmidazolium, 200 μM [β-32P]ATP (300-500 c.p.m./pmol) and 40S ribosomes (2 mg ml-1 final concentration).

Quantification of PHAS-1-eIF4E and eIF4E-eIF4G Complexes.

Each frozen muscle was placed in a liquid-nitrogen-chilled porcelain mortar to which 0.5 ml of the following homogenization buffer (pH 7.0) was added: 50 mM NaF, 50 mM β-glycerophosphate, 0.1 mM microcystin-LR, 10 mM potassium phosphate, 10 mM MgCl2, 1 mM EDTA, 1 mM EGTA, 1 mM sodium vanadate, 0.1% Tween 20, 1 mM PMSF, 1 mM benzamidine, 10 μg ml-1 leupeptin and 10 μg ml-1 aprotinin. The tissue was ground into powder, which was placed in a glass homogenization tube and left to thaw while being homogenized with a Teflon pestle driven at 1,000 r.p.m. The homogenates were centrifuged at 10,000 g for 30 min and the protein concentration of the supernatant fractions was determined. eIF4E-bound forms of PHAS-1/4E-BP1 and eIF4G were isolated by affinity-purification of the complexes with m7GTP-Sepharose (Pharmacia)16. In brief, samples (1 mg protein/500 μl) of extract were incubated with resin (30 μl) packed beads for 30 min at 4° C. The beads were then washed three times (1 ml buffer per wash) before proteins were eluted with SDS-sample buffer. PHAS-1/4E-BP1 and eIF4E were detected with the antibodies and immunoblotting procedures described previously(Lin, T. -A. & Lawrence, J. C. Jr J. Biol. Chem. 271, 30199-30204 (1996) eIF4G was detected by immunoblotting with an antibody generated against a peptide (CQKEFEKDKDDDEVFEKKQKEMDEA; single-letter amino acid abbreviations) corresponding to a sequence in eIF4G. In brief, the rabbits were immunized with peptide (0.5 mg peptide per injection) that had been conjugated to keyhole limpet haemocyanin, and boosted with the conjugate at monthly intervals. Antibodies were affinity-purified with a resin prepared by coupling the peptide via its amino-terminal cysteine to SulfoLink (Pierce). After the column had been washed, antibodies were eluted at pH 2.7, neutralized immediately and isolated after a final purification step with Protein A-agarose.

Data Analysis.

Muscle fibre size was obtained from digitally imaged serial cross-sections of cryostat-sectioned muscle. Individual muscle fibres were outlined and cross-sectional area was determined with a computerassisted image analysis system (MetaMorph™; Universal Imaging Corporation). All data are expressed as means ±s.e.m. (represented as error bars). A one-way analysis of variance with Fisher's post-hoc correction for multiple paired comparisons was used for comparisons between groups. Statistical significance was set at P<0.05.

### Example 8

### Effects of Differentiation on Myotubes

The idea that calcineurin has a role in skeletal muscle hypertrophy was based largely on findings that either cyclosporin A (CsA; a calcineurin inhibitor) or dominant-negative forms of calcineurin could block IGF-1-induced hypertrophy in muscle cultures (Musaro, A., McCullagh, K. J., Naya, F. J., Olson, E. N. & Rosenthal, N. Nature 400, 581-585 (1999); Semsarian, C., Sutrave, P., Richmond, D. R. & Graham, R. M. Biochem. J. 339, 443-451 (1999)). However, these findings might have resulted from an inhibition of myoblast differentiation and fusion, as opposed to direct inhibition of muscle hypertrophy, because the calcineurin blockers were administered to undifferentiated myoblasts, before fusion, and because calcineurin has since been shown to be required for myoblast differentiation. (Musaro, A., McCullagh, K. J., Naya, F. J., Olson, E. N. & Rosenthal, N. Nature 400, 581-585 (1999); Semsarian, C., Sutrave, P., Richmond, D. R. & Graham, R. M. Biochem. J. 339,443-451 (1999); 8. Rommel, C. et al. Science 286, 1738-1741 (1999); Friday, B. B., Horsley, V. & Pavlath, G. K. J. Cell Biol. 149, 657-666 (2000)). The possibility that the PI(3)K/Akt pathway accounts for the hypertrophic actions of IGF-1 has not been explored adequately, although IGF-1 has been shown to activate this pathway (Dudek, H. et al. Science 275, 661-665 (1997)).

To distinguish between effects on myoblast differentiation and those on the trophic state of existing myofibres, we exploited the C2C12 myoblast differentiation system. C2C12 myoblasts proliferate until they reach confluence; then, in serum-poor medium, the myoblasts fuse into multi-nucleated myotubes. Differentiated myotubes are predominant by 2 days after fusion (D2 myotubes) and survive for another 3 days. D2 myotubes can be treated with 10 ng ml-1 IGF-1, resulting in hypertrophy, as measured by a comparison of myotube diameters, or by an increase in protein content per myotube (Rommel, C. et al. Science 286, 1738-1741 (1999)). In contrast, if pre-differentiation myoblasts are treated with IGF-1, the result is an increase in proliferation (. Svegliati-Baroni, G. et al. Hepatology 29, 1743-1751 (1999); Yu, H. & Berkel, H. J. LA State Med. Soc. 151, 218-223 (1999)). Thus, myoblasts and myotubes respond differently to growth-factor stimuli, demonstrating the need to distinguish between pre-differentiation and post-differentiation effects. IGF-1-induced myotube hypertrophy, as measured by an increase in diameter and an increase in total protein, is normally accompanied by an increase in nuclei within the myofibres. However, hypertrophy can be uncoupled from DNA replication, as myotube diameter and total protein still increase as a result of stimulation with IGF-1 in the presence of cytosine arabinoside (Ara-C), an inhibitor of DNA synthesis.

### Example 9

### Direct Addition of IGF-1 to Myotubes

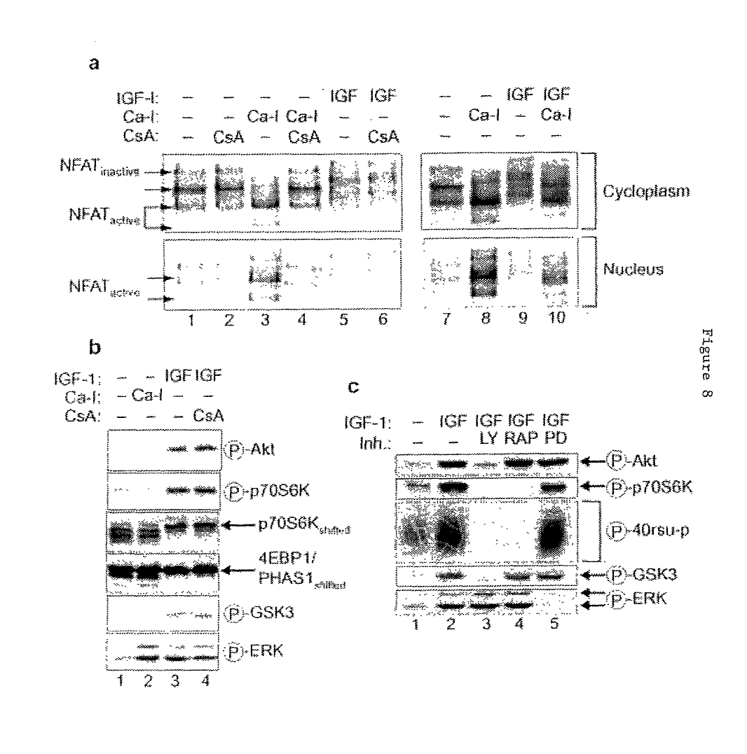

As a first step towards examining the roles of the calcineurin/NFAT and PI(3)K/Akt pathways during IGF-induced myotube hypertrophy, we added IGF-1 to differentiated myotubes and determined whether these pathways were activated. Calcineurin is a protein phosphatase that is activated by increases in intracellular calcium levels, which in turn dephosphorylates the NFAT transcription factors; this dephosphorylation results in the translocation of NFATs to the nucleus and subsequent gene transcription. NFAT dephosphorylation and nuclear translocation are therefore markers of calcineurin activation (FIG. 7, right). For markers of activation of the PI(3)K/Akt pathway, the phosphorylation of Akt itself or of downstream targets such as p70S6 kinase (p70S6K), PHAS-1/4E-BP1 or GSK3 were used (FIG. 7).

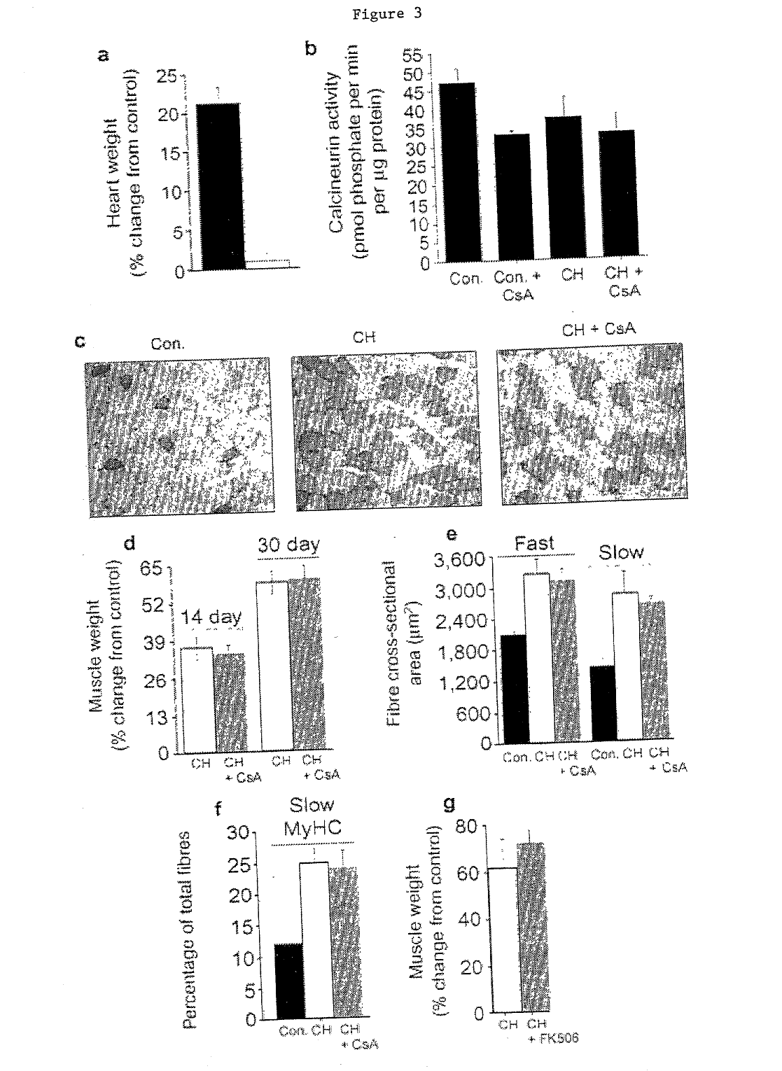

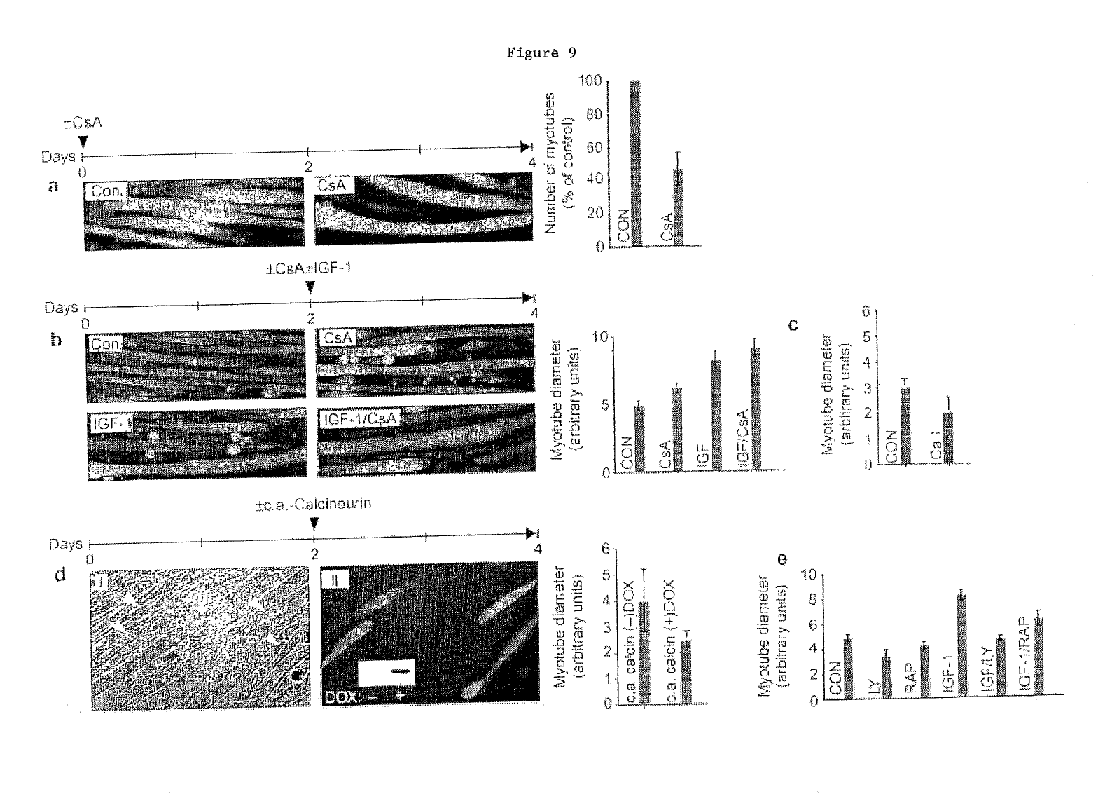

Doses of IGF-1 sufficient to cause hypertrophy (see below) did not cause a downshift in dephosphorylation or a nuclear translocation of NFAT, but instead caused a hyperphosphorylation of NFAT (FIG. 8a), indicating that IGF-1 might be inhibiting calcineurin activity. As a control to show that the calcineurin pathway could be activated in myotubes, treatment with a calcium ionophore (a standard method of activating calcineurin) resulted in a dephosphorylation-induced downshift in NFAT, gel mobility (FIG. 8a, top) and nuclear translocation (FIG. 2a, bottom) of the NFAT isoform previously implicated in myotube hypertrophy, NFAT-C1 (Goldspink, G. J. Anat. 194, 323-334 (1999); Florini, J. R., Ewton, D. Z. & Coolican, S. A. Endocr. Rev. 17,481-517 (1996); Musaro, A., McCullagh, K. J., Naya, F. J., Olson, E. N. & Rosenthal, N. Nature 400, 581-585 (1999)). The calcineurin inhibitor CsA blocked the calcium-ionophore-induced dephosphorylation and translocation of NFATC-1 (FIG. 8a, lane 4). Further supporting the notion that IGF-1 might antagonize calcineurin signaling, simultaneous treatment with IGF-1 and calcium ionophore blunted the ability of the calcium ionophore to activate NFAT-C1 (FIG. 8a, lanes 7-10).

Although IGF-1 could not activate the calcineurin pathway in myotubes, it induced the phosphorylation of many of the defined downstream targets of the I(3)K/Akt/mTOR pathway (FIG. 8b, lane 3). Consistent with the notion that this IGF1-induced activation of the PI(3)K/Akt pathway was independent of the calcineurin pathway was the observation that none of these markers of Akt activation was inhibited by using CsA as a calcineurin blocker (FIG. 8b, lane 4), and none could be induced with calcium ionophore as a calcineurin activator (FIG. 8b, lane 2). Interestingly, the only shared signaling molecule induced by activation both with IGF-1 and with calcineurin was extracellular signal-regulated kinase (ERK) (FIG. 8b, bottom); previous evidence indicates that in myotubes the ERK pathway acts counter to the PI(3)K/Akt pathway and inhibits the hypertrophic response (Rommel, C. et al. Science 286, 1738-1741 (1999)).

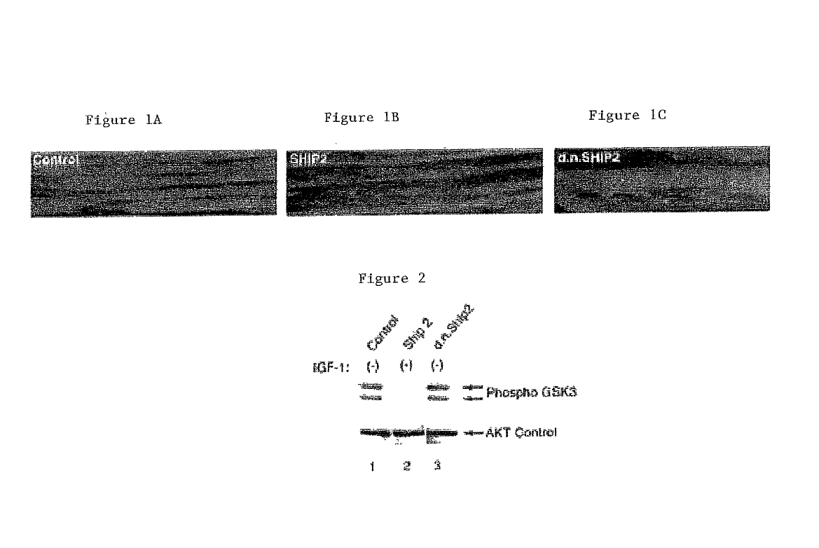

Pharmacological inhibitors of the PI(3)K/Akt/mTOR pathway were subsequently used in phenotypic assays of hypertrophy. To confirm the specificity of these inhibitors, we characterized them with regard to the biochemical activations induced by IGF-1. The PI(3)K inhibitor LY294002 (FIG. 7) blocked all measured activations in the pathway beginning with Akt, but did not affect ERK activation (FIG. 8c, lane 3). The more downstream inhibitor rapamycin (FIG. 1), which acts on mTOR, did not affect upstream Akt activation nor a branch of this pathway represented by GSK-3, but did block the phosphorylation of two known mediators of protein synthesis downstream of mTOR, p70S6K and PHAS-1/4E-BP1 (FIG. 8c, lane 4). The above correlative findings supported the possibility that IGF-1 promotes muscle hypertrophy through the PI(3)K/Akt pathway in a calcineurin-independent manner.

### Example 10

### Inhibition of the Calcineurin and PI(3)K/Akt Pathways

To obtain more direct evidence, we next used the pharmacological inhibitors of the calcineurin and PI(3)K/Akt pathways in assays of IGF-1-induced myotube hypertrophy. Further, we introduced gene-expression vectors into the myotubes that induced the expression of either activators or dominant-negative blockers of these pathways. Hypertrophy was quantified by measuring myotube diameter; increases in diameter were correlated with total protein increases in IGF-1-treated cultures (data not shown), but unfortunately we could not use the independent measure of change in total protein in some of the following experiments because only a fraction of the fibres expressed the transfected gene of interest.

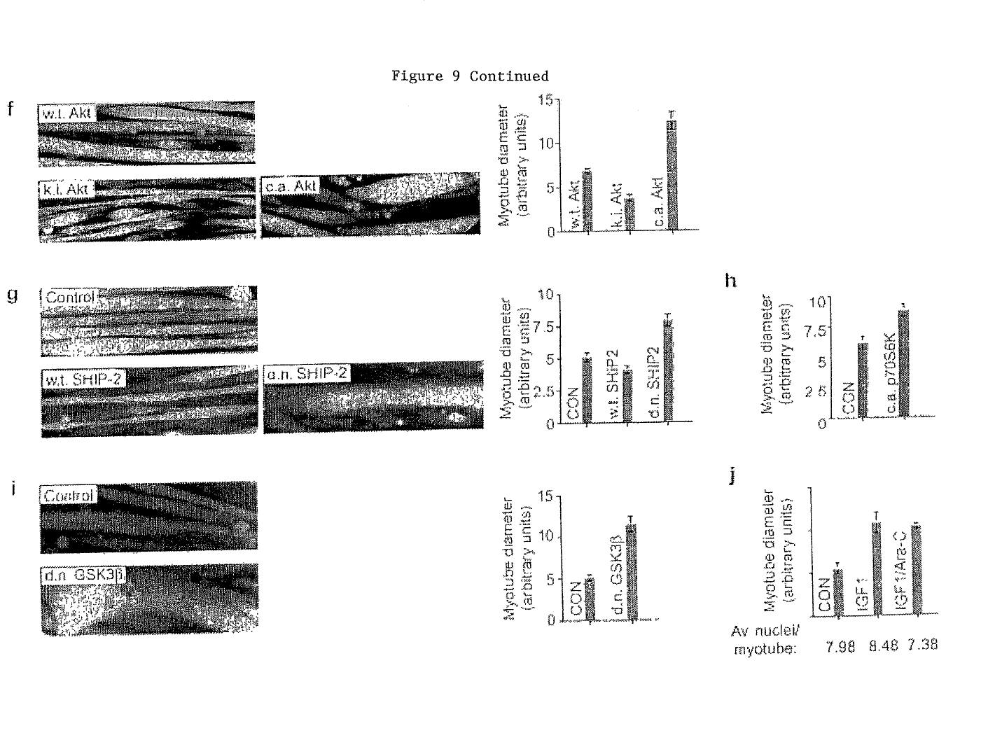

Addition of the calcineurin inhibitor CsA at a pharmacologically efficacious dose (as shown in FIG. 8a) to pre-differentiation myoblasts resulted in significantly fewer myotubes, which is consistent with a requirement for calcineurin during differentiation (FIG. 9a). To establish a requirement for calcineurin in hypertrophy, as opposed to differentiation, CsA was added to already-differentiated D2 myotubes; in this setting, CsA did not prevent IGF-induced hypertrophy—instead, there was a potentiation of hypertrophy by CsA in both control and IGF-treated myotubes (FIG. 9b). Treatment of differentiated myotubes with a calcium ionophore that iochemically activates calcineurin (FIG. 8a), or expression in myotubes of a doxycycline-regulatable gene expression vector, permitting the doxycycline-inducible expression of a constitutively active form of calcineurin only after myotube formation (see FIG. 9d, inset), led to thinner myotubes (FIGS. 9c, d), indicating that neither the pharmacological nor the genetic activation of the calcineurin pathway is sufficient to cause hypertrophy in differentiated myotubes.

We next blocked the PI(3)K/Akt pathway pharmacologically, to see whether, in addition to being correlated with hypertrophy (FIGS. 8b, c), the PI(3)K/Akt pathway was required for hypertrophy. Inhibition of PI(3)K with LY294002 led to a mild atrophy of control myotubes and completely blocked IGF-induced hypertrophy (FIG. 9e). Among the downstream effectors of the PI(3)K/Akt pathway, the mTOR kinase is an important regulator of protein synthesis (FIG. 7); pharmacological blockade with the mTOR inhibitor rapamycin was less effective than PI(3)K blockade but significantly blunted IGF-induced hypertrophy (FIG. 9e); the more marked effects seen with upstream inhibition of the PI(3)K/Akt/mTOR pathway suggested that multiple branches of this pathway might contribute to hypertrophy (see below) and that mTOR is not required for all of these. As a specificity control for these anipulations, an inhibitor of the ERK pathway known as PD98059 did indeed block ERK activation without affecting steps in the PI(3)K/Akt/mTOR pathway (FIG. 8c); the PD98059 inhibitor did not block phenotypic hypertrophy (data not shown).

### Example 12

### Genetic Manipulation of the PI(3)K/Akt Pathway

To confirm the role of the PI(3)K/Akt pathway independently, and to determine whether it might be sufficient to cause hypertrophy, we exploited a genetic approach. The first genetic construct permitted the expression of a kinase-inactive Akt that has been shown to block endogenous Akt activity when overexpressed; the second construct permitted the expression of a constitutively active Akt (Eves, E. M. et al. Mol. Cell. Biol. 18, 2143-2152 (1998)). These two constructs behaved as would be predicted if the Akt pathway were sufficient for hypertrophy: the kinase-inactive Akt resulted in thinner myotubes, whereas the constitutively active Akt caused phenotypic hypertrophy (FIG. 9f). The third and fourth constructs permitted the indirect regulation of the PI(3)K/Akt pathway by way of the inositol phosphatase SHIP2 (FIG. 7). Because Akt activity depends on levels of phosphatidylinositol-3,4,5-trisphosphate PtdIns(3,4,5)P3, overexpression of the inositol phosphatase SHIP2 should attenuate the Akt pathway by decreasing PtdIns(3,4,5)P3 levels (as has been shown for SHIP1, whereas overexpression of a dominant-negative mutant of SHIP2 should promote the Akt pathway by increasing PtdIns(3,4,5)P3 levels (as shown for SHIP1 (Aman, M. J., Lamkin, T. D., Okada, H., Kurosaki, T. & Ravichandran, K. S. J. Biol. Chem. 273,33922-33928 (1998); Carver, D. J., Aman, M. J. & Ravichandran, K. S. Blood 96, 1449-1456 (2000); Liu, Q. et al. Genes Dev. 13, 786-791 (1999); Jacob, A., Cooney, D., Tridandapani, S., Kelley, T. & Coggeshall, K. M. J. Biol. Chem. 274,13704-13710 (1999)).

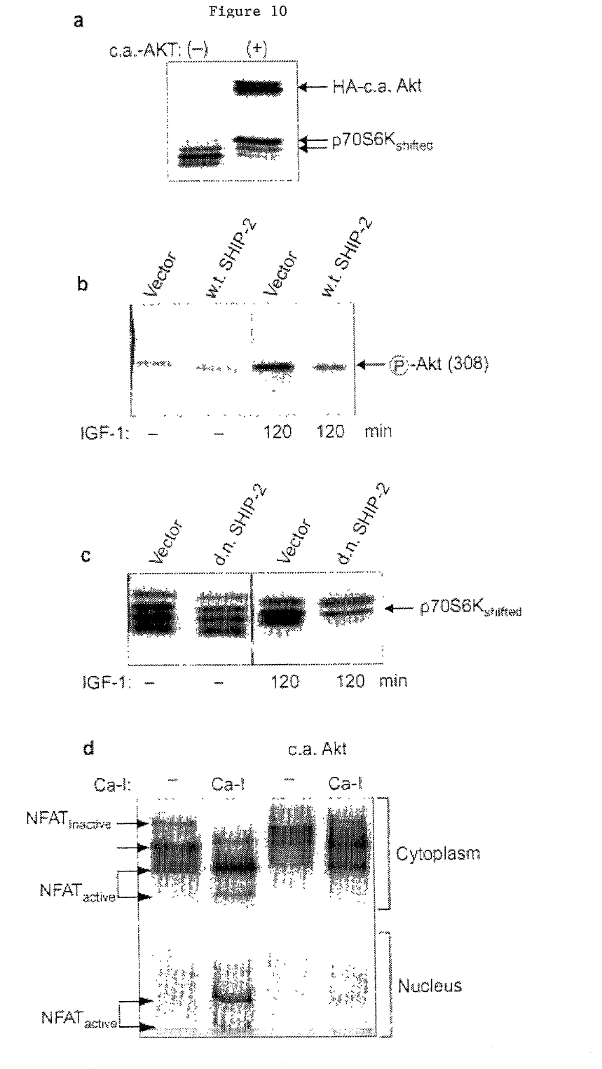

Overexpression of wild-type SHIP2 resulted in atrophic myotubes (FIG. 9g), whereas expression of the dominant-negative mutant of SHIP2 induced hypertrophy (FIG. 9g). Thus, indirect regulation of Akt, by manipulation of SHIP2 activity, resulted in phenotypes consistent with the direct manipulation of Akt. We confirmed that the gene expression constructs were affecting intracellular signaling as expected: overexpression of constitutively active Akt cause d activation of p70S6K (FIG. 10a); overexpression of wild-type SHIP2 accelerated inactivation of Akt (FIG. 10b); overexpression of the dominant-negative form of SHIP2 resulted in IGF-induced hyperphosphorylation of p70S6K (FIG. 10c).

The above genetic approach confirmed the unexpected and surprising finding noted above, that IGF-1 inhibits the calcineurin/NFAT pathway by promoting NFAT-C1 hyperphosphorylation, and further demonstrated that IGF-1 acts in this manner via the Akt pathway: we found that the constitutively active Akt resulted in the hyperphosphorylation of NFAT-C1 (FIG. 10d, lane 3) and blocked its activation and nuclar translocation (FIG. 10d, lane 4).

We next examined the PI(3)K/Akt/mTOR pathway genetically, by making use of a constitutively active form of p70S6K. This construct caused hypertrophy (FIG. 9h) but was less potent than the constitutively active Akt construct (FIG. 9f), which is consistent with the idea that p70S6K might be only one of several subpathways (including, for example, the 4E-BP1/PHAS-1 subpathway) downstream of Akt and mTOR. The smaller effects seen by more downstream inhibition or activation of the PI(3)K/Akt/mTOR pathway suggests that Akt targets in addition to mTOR are important in hypertrophy. One obvious such target is GSK3 (FIG. 7), whose activity is inhibited after phosphorylation by Akt; because GSK3 normally acts to inhibit the translation initiation factor eIF2B, blockade of GSK3 by Akt might promote translation initiation and protein synthesis, and thus might contribute to hypertrophy. To examine this possibility directly, we expressed a dominantnegative form of GSK3β in myotubes and found that it caused profound myotube hypertrophy (FIG. 9i). Together with the above findings that IGF-1 results in phosphorylation of GSK3 via a mechanism that is blocked by the PI(3)K inhibitor LY294002 (FIG. 8c) but not by rapamycin (FIG. 8c), the results with the dominant-negative GSK3 strongly argue that this represents a downstream target of the IGF/PI(3)K/Akt pathway that has a crucial role in regulating muscle hypertrophy in an mTOR-independent manner (FIG. 7).

### Discussion of Examples 8-12

In this study we explored the signaling pathways by which IGF-1 mediates muscle hypertrophy in vitro. We found that PI(3)K/Akt pathway activation occurs during IGF-induced hypertrophy, and showed further that this pathway is necessary for IGF-1-mediated c.a.-AKT: (−) (+) hypertrophy by demonstrating that its inhibition prevents hypertrophy. We further report that Akt pathway activation is sufficient to cause hypertrophy. Akt activates multiple downstream signaling pathways, and our findings define at least two major downstream targets of Akt in the hypertrophic response (FIG. 7). We first circumstantially implicated the Akt/mTOR and Akt/GSK3 subpathways in the hypertrophic response by showing that they were biochemically regulated by IGF-1 and Akt. We then demonstrated required roles for both the mTOR and GSK3 subpathways during hypertrophy by exploiting either pharmacological or genetic means. However, we cannot rule out the possibility that other Akt targets might also be involved.

While our findings seem to confirm a crucial role for Akt-dependent pathways in the IGF-1-induced hypertrophy response, our efforts seem to have ruled out a required role for the calcineurin/NFAT pathway after differentiation. In mature myotubes this pathway is not activated by IGF-1, inhibition of calcineurin by CsA does not blunt IGF-induced hypertrophy, and constitutive activation of calcineurin does not cause hypertrophy. In contrast, we find that calcineurin activity is necessary for myotube differentiation, which is consistent with a recent study (Friday, B. B., Horsley, V. & Favlath, G. K. J. Cell Biol. 149, 657-666 (2000)). In reconciling these findings with two previous reports indicating that calcineurin was crucial for IGF-1-mediated hypertrophy, it should be noted that those studies added CsA as a calcineurin inhibitor before the differentiation of myoblasts into myotubes, which is consistent with the possibility that the lack of hypertrophy seen in those studies was a consequence of an inhibition of differentiation. (Musaro, A., McCullagh, K. J., Naya, F. J., Olson, E. N. & Rosenthal, N. Nature 400, 581-585 (1999); Semsarian, C., Sutrave, P., Richmond, D. R. & Graham, R. M. Biochem. J. 339, 443-451 (1999)). It is certainly possible that at different points in the development process, muscle fibre size can be regulated both by the fusion of additional satellite cells into a myofibre and by enhanced protein synthesis within a myofibre. We show here that the addition of Ara-C, an inhibitor of DNA replication, does not inhibit IGF-1-mediated hypertrophy; myotube hypertrophy can therefore be uncoupled from DNA replication, because myotube diameter and total protein still increase as a result of stimulation with IGF-1 (FIG. 9j). In terms of the role of the calcineurin/NFAT pathway in hypertrophy, our data suggest that calcineurin might even oppose the hypertrophic response in the mature myotube, because IGF-1 and Akt antagonize the calcineurin-mediated dephosphorylation and translocation of NFAT-C1.

Our findings are consistent with previous findings in Drosophila, in which overexpression of the insulin receptor substrate IRS-1 or Akt or p70S6K were sufficient to cause hypertrophy of the cells in which they were expressed. The role of the Akt pathway in mammalian myotubes might be analogous to that in Drosophila, in which the Akt pathway promotes hypertrophy by way of pro-synthetic pathways and can be separated from proliferation effects. Furthermore, the findings reported extend our in vitro findings and confirm their physiological relevance, by defining a crucial role for Akt signaling in a variety of muscle hypertrophy responses in vivo. Although further work is needed to understand how the Akt pathway contributes to hypertrophy, the immediate therapeutic ramifications of these findings indicate that pharmacological inhibitors of either SHIP2 or GSK3, as well as pharmacological activators of Akt, mTOR or p70S6K, could provide therapeuticbenefit for muscle atrophy.

### Materials and Methods for Examples 8-12

Cell Culture and Myotube Analysis.

C2C12 myoblasts were cultured and transfected as described (Glass, D. J. et al. Cold Spring Harb. Symp. Quant. Biol. 57, 53-62 (1992)). Flow cytometry and cell sorting were performed on a Cytomation MoFlo (Fort Collins, Colorado) high-speed cell sorter (FACS) (Rommel, C. et al. Science 286, 1738-1741 (1999)). Myoblasts were fused into myotubes at confluence, by shifting the medium to DMEM+2% horse serum. Myotube diameter was quantified as follows: 10 fields were chosen randomly, and approximately 10 myotubes were measured per field. The average diameter per myotube was calculated as the mean of ten measurements taken along the length of the myotube. For the constitutively active Akt, kinaseinactive Akt, wild-type Akt, wild-type SHIP2, dominant-negative SHIP2, constitutively active p70S6K and dominant-negative GSK3β constructs, transfected myotubes were determined by the co-expression of enhanced green fluorescent protein (EGFP; see below for construct details); thus, only transfected myotubes were assessed for hypertrophy. For the tetracycline-inducible calcineurin, myotubes expressing calcineurin were identified by EGFP expression, and comparisons were made between EGFP-positive and EGFP-negative myotubes. Photographs shown in FIG. 3 were all taken at a magnification of •200.

Growth Factors, Pharmacological Agents and Antibodies

Before the preparation of cell lysate, serum-starved cells were treated with R3-IGF-1

(IGF-1: 10 ng ml-1; Sigma), A23187 (0.1-10 μM in dimethylsulphoxide (DMSO); Calbiochem), LY294002 (10 μM in DMSO; Calbiochem), rapamycin (20 ng ml-1 in DMSO; Calbiochem), CsA (5 μM in ethanol; Calbiochem), PD98059 (10 μM in DMSO; Calbiochem) or cytosine-β-D-arabinofuranoside (Ara-C, 10 μM in water; Sigma). Protein analysis of total cell lysates was conducted as described8. The antibodies used were anti-phospho-Erk1/2 (Thr 202/Tyr 204; NEB); anti-phospho-Akt (Ser 473; NEB); anti-phospho- GSK3 (Ser 9/21; NEB); anti-phospho-p70S6K (Thr 389; NEB); anti-phospho-PHAS-1/4E-BP1 (Thr 65; NEB); anti-Erk1/2 (UBI), anti-Akt (NEB), anti-p70S6K (C-18; Santa Cruz), anti-PHAS-1/4EBP1 (Zymed) and anti-NFAT-C1 (7A6; Santa Cruz).

Protein Analysis

Immunoprecipitations and immunoblotting were performed as described. Proteins were detected with the chemiluminescence detection system Renaissance; NEN). To measure the enzymatic activity of p70S6K an immunocomplex kinase assay was performed as described (Azpiazu, I., Saltiel, A. R., DePaoli-Roach, A. A. & Lawrence, J. C. J. Biol. Chem. 271, 5033-5039 (1996)).

Molecular Biology and Selection of Inducible-Expression Constructs

Constitutively active calcineurin (carboxy-terminal deletion mutant encoding amino acid residues 1-398 of calcineurin) tagged with the Flag epitope ([EYKEEEK]2) at the carboxy terminus was generated by the polymerase chain reaction from mouse skeletal muscle complementary DNA (Marathon- Ready; Clontech) and was subsequently subcloned into a tetracycline-inducible internal ribosomal entry site (IRES) bicistronic expression vector (pTRE-Flag-c.a.-calcineurin-IRESEGFP). The reverse tetracycline-controlled transcriptional activator (rtTA) was fused at its C terminus to enhanced blue fluorescence protein (EBFP; Clontech) and subcloned into an expression vector containing the muscle creatine kinase (MCK) promoter8. The tetracycline-responsive vector encoding constitutively active calcineurin and EGFP on the same transcript (as a constitutively active calcineurin-IRESEGFP cassette) was stably transfected into an MCK-rtTAEBFP cell line. Myoblasts harvested after FACS analysis were treated 48 h after the induction of myogenic differentiation with 2 μg ml-1 doxycycline (from a 10 mg ml-1 stock solution in water; SIGMA). At day 4 of differentiation, cell lysates were prepared as described8 and calcineurin was immunoprecipitated with an anti-Flag antibody (Sigma) followed by immunoblot analysis with anti-Flag. Constitutively active Akt was as described previously; it was expressed in a vector containing the MCK promoter and an IRES-EGFP cassette. The kinase-inactive Akt was a gift from the Tsichlis laboratory and was cloned into the same MCK-IRES-EGFP vector. The constitutively active form of p70S6K was a gift from John Blenis's laboratory and was also cloned into the same MCK-IRES-EGFP vector; the kinase activity of the constitutively active p70S6K was determined by transiently transfecting the construct, a wtp70S6K construct and a negative-control vector construct into COS cells, starving those cells and determining the kinase activity as described (data not shown). Human SHIP2 was cloned from an Origene library. The dominant-negative mutant of SHIP2 contained a D690A mutation. SHIP2 was tagged with the haemagglutinin epitope and cloned into the MCK-IRES-EGFP vector.

Although the foregoing invention has been described in some detail by way of illustration and example, it will be readily apparent to those of ordinary skill in the art that certain changes and modifications may be made to the teachings of the invention without departing from the spirit or scope of the invention.

We claim:

1. A method of inhibiting atrophy or inducing hypertrophy in skeletal muscle cells comprising treating the cells with an inhibitor of the SHIP2 pathway.

2. The method of claim 1 wherein the inhibitor is an inhibitor of SHIP2.

3. The method of claim 1 wherein the inhibitor of SHIP2 causes phosphorylation of Akt.

4. The method claim 1 wherein the inhibitor of the SHIP2 pathway is an activator of Akt.

5. A method of inhibiting atrophy in skeletal muscle cells comprising treating the cells with a muscle tissue-specific activator of the PI3K/Akt pathway.

6. The method of any one of claims 1, 2, 3, 4, or 5 wherein the skeletal muscle cells are in a vertebrate animal having an atrophy-inducing condition.

7. The method of claim 6 wherein the vertebrate animal is a chicken, rodent, rabbit, dog, cat, cow, horse, pig, sheep, primate, or human.

8. The method of claim 6 wherein the vertebrate animal is treated prior to exposure to or onset of the atrophy-inducing condition.

9. The method of claim 6 wherein the atrophy-inducing condition is immobilization, denervation, starvation, nutritional deficiency, metabolic stress, diabetes, aging, muscular dystrophy, AIDS/HIV infection, cancer, bed rest or myopathy.

10. A method of identifying a test agent that inhibits muscle atrophy or induces hypertrophy comprising:
(a) obtaining cells that express the following:
1) SHIP2;
2) an Akt substrate/reporter construct capable of measuring Akt pathway activation;
(b) subjecting the cells to a test agent;
(c) measuring the amount of Akt pathway activation, wherein activation of the Akt pathway is used to identify a test agent that inhibits atrophy.

11. The method of claim 10 wherein the cells are fibroblasts.

12. The method of claim 10 wherein the cells are muscle cells.

13. The method of claim 12 wherein the muscle cells are myoblasts.

14. The method of claim 13 wherein the myoblasts are C2C12 cells.

15. A method of reducing muscle atrophy or inducing muscle hypertrophy in skeletal muscle cells comprising treating the cells with an activator of the PI3K/Akt pathway.

16. The method of claim 15 wherein the activator is a specific activator of Akt.

17. The method of claim 15 wherein the activator causes phosphorylation of Akt substrates, such as mTOR, forkhead, or GSK3.

18. A method of reducing muscle atrophy or inducing muscle hypertrophy in skeletal muscle cells comprising treating the cells with a muscle tissue-specific activator of the PI3K/Akt pathway.

19. A method of reducing muscle atrophy or inducing muscle hypertrophy in skeletal muscle cells comprising treating the cells with a muscle tissue-specific inhibitor of the SHIP2 pathway.

20. A method of reducing muscle atrophy or inducing muscle hypertrophy in skeletal muscle cells comprising treating the cells with an inhibitor of the SHIP2 pathway.

21. A method of reducing muscle atrophy or inducing muscle hypertrophy in skeletal muscle cells comprising treating the cells with an inhibitor of SHIP2.

22. The method of any one of claims 15, 18, 19, 20, or 21, wherein the skeletal muscle cells are within a vertebrate animal.

23. The method of claim 22 wherein the vertebrate animal is a chicken, rodent, rabbit, dog, cat, cow, horse, pig, sheep, primate, or human.

24. A cell comprising:
1) SHIP2;
2) an Akt substrate/reporter construct capable of measuring Akt substrate activation.

25. A SHIP2 antagonist for use in a method of inhibiting atrophy, inducing hypertrophy, activating the Akt pathway, interfering with the calcineurin pathway, or modulating Akt expression or activity.

26. A method of screening compounds useful for the treatment of muscle atrophy or detecting atrophy and related diseases and disorders comprising contacting a muscle cell expressing SHIP2 with a compound and detecting a change in the SHIP2 protein activity or the Akt pathway.

27. The method of claim 26 wherein the change is measured by PCR, Taqman PCR, phage display systems, gel electrophoresis, yeast-two hybrid assay, Northern or Western analysis, immunohistochemistry, a conventional scintillation camera, a gamma camera, a rectilinear scanner, a PET scanner, a SPECT scanner, a MRI scanner, a NMR scanner, or an X-ray machine.

28. The method of claim 26 where in the change in SHIP2 protein activity is detected by detecting a change in the interaction of SHIP2 with one or more proteins, by detecting a change in the interaction of Akt with another protein, or by detecting a change in the level of one or more of the proteins in the Akt pathway.

29. The method of claim 26 wherein the muscle cell is of skeletal muscle origin.

30. The method of claim 26 wherein the muscle cells are cultured cells.

31. The method of claim 26 wherein the muscle cells are obtained from a transgenic organism.

32. The method of claim 31 wherein the transgenic organism includes, but is not limited to a mouse, rat, rabbit, sheep, cow or primate.

33. The method of claim 26 wherein the muscle cells are within a transgenic organism.

34. The method of claim 33 wherein the transgenic organism includes, but is not limited to a mouse, rat, rabbit, sheep, cow or primate.

35. A method of detecting muscle atrophy in an animal comprising measuring SHIP2 in a patient sample.

36. A method of inhibiting atrophy or inducing hypertrophy by modulating SHIP2.

37. A method of treating illnesses, syndromes or disorders associated with muscle atrophy comprising administering to an animal a compound that modulates SHIP2 or the Akt pathway such that symptoms are alleviated.

38. The method of claim 37 such that the animal is a mammal.

39. The method of claim 37 such that the mammal is a human.

40. A method of identifying a test agent capable of inhibiting muscle atrophy or inducing muscle hypertrophy in vitro comprising:
(a) contacting a mixture containing SHIP2 and phosphatidylinositol 3,4,5-trisphosphate with a test agent; and
(b) measuring the ability of SHIP2 to mediate the conversion of phosphatidylinositol 3,4,5-trisphosphate.

41. The method of claim 40 wherein such measuring is accomplished either by measuring the release of the phosphate at the “5” position, or by determining the amount of residual phosphatidylinositol 3,4,5-trisphosphate or measuring binding of a phosphotidylinositol-3,4,5-trisphosphate-specific agent to phosphotidylinositol-3,4,5-trisphosphate.

42. The method of claims 41 such that the phosphotidylinositol-3,4,5-trisphosphate-specific agent is Akt.

43. The method of claim 40 wherein such measuring is accomplished by fluorescence, PCR, Taqman PCR, phage display systems, gel electrophoresis, yeast-two hybrid assay, Northern or Western analysis, immunohistochemistry, a conventional scintillation camera, a gamma camera, a rectilinear scanner, a PET scanner, a SPECT scanner, a MRI scanner, a NMR scanner, or an X-ray machine

44. The method of claim 40 wherein such test agent is a carbohydrate, a lipid, a protein, a salt, a nucleic acid, or a small molecule.
